(19) United States
(12) Patent Application Publication
(10) Pub. No.: US 2024/0299307 A1
(43) Pub. Date: Sep. 12, 2024

(54) Extended-release compositions comprising atomoxetine

(71) Applicant: Tulex Pharmaceuticals, Inc., Cranbury, NJ (US)

(72) Inventors: Yu-Hsing Tu, West Windsor, NJ (US); Shanmuka Harish Chalamuri, Plainsboro, NJ (US); Kalyan Kathala, Monroe, NJ (US); Ashok Perumal, Monmouth Junction, NJ (US); James A. Lee, West Windsor, NJ (US)

(73) Assignee: Tulex Pharmaceuticals, Inc., Cranbury, NJ (US)

(21) Appl. No.: 18/549,944

(22) PCT Filed: Feb. 15, 2022
(86) PCT No.: PCT/US2022/016377

§ 371 Date: Sep. 11, 2023

Related U.S. Application Data
(60) Provisional application No. 63/158,511, filed on Mar. 9, 2021.

(51) Int. Cl. A61K 9/50; A61K 31/138

(52) U.S. Cl. CPC A61K 9/5047; A61K 9/5073; A61K 31/138

(57) ABSTRACT

The present disclosure provides extended release atomoxetine compositions suitable for once-daily administration. The compositions comprise a core comprising atomoxetine or a pharmaceutically acceptable salt thereof; and a functional coat/extended release coat over the core. The extended release compositions of the disclosure provide a lag time of at least about 30 minutes during which compositions release less than or equal to 20% of atomoxetine or the pharmaceutically acceptable salt thereof, based on the total weight of atomoxetine or a pharmaceutically acceptable salt thereof present in the composition, measured in 900 mL of a dissolution medium comprising 0.05 M pH 6.8 buffer, using USP Apparatus II (paddle) at 50 rpm and 37° C. In certain embodiments, the compositions of the disclosure release less than or equal to 40% of atomoxetine or the pharmaceutically acceptable salt thereof, based on the total weight of atomoxetine or a pharmaceutically acceptable salt thereof present in the composition, within 2 hours of coming in contact with 900 mL of a dissolution medium comprising 0.05 M pH 6.8 buffer, measured using USP Apparatus II (paddle) at 50 rpm and 37° C. The atomoxetine compositions of the disclosure provide extended release for at least about 8 hours and are suitable for once-a-day administration.

### 1. RELATED APPLICATIONS

This application claims priority to U.S. Provisional Application No. 63/158,511, filed Mar. 9, 2021.

### 2. TECHNICAL FIELD

The present disclosure provides extended release (ER) atomoxetine (ATO) compositions suitable for once daily administration. The compositions of the disclosure provide extended release profile of atomoxetine in therapeutic range by reducing dose related peak-to-trough fluctuations in plasma concentrations of atomoxetine. The compositions of the disclosure improve tolerability and patient compliance by reducing adverse effects associated with high peak-to-trough fluctuations. The ER atomoxetine compositions of the disclosure include pellets suitable for dosing in tablets, capsules, sachets, administration with feeding tube, and as sprinkles on food.

### 3. BACKGROUND

Attention-deficit/hyperactivity disorder (ADHD) is a central nervous system disorder characterized by hyperactivity and difficulties controlling impulses and sustaining attention. It occurs in 3% to 7% of school-age children in the United States (Diagnostic and Statistical manual of Mental Disorders, 4^{th} ed. Washington DC: American Psychiatric Association; 2000). Although, ADHD symptoms tend to improve with age, an estimated 4% of the adult population has ADHD.

Atomoxetine, a highly specific inhibitor of the parasynaptic norepinephrine transporter, is an FDA-approved treatment for ADHD in adults and children. Atomoxetine is not a psychostimulant, which sets it apart from the common medications used for ADHD, e.g., methylphenidate and mixed amphetamines. Atomoxetine is the R(−) isomer of tomoxetine; the chemical name for atomoxetine is ((−)-N-methyl-3-phenyl-3-(o-tolyloxy)-propylamine.

Atomoxetine and its salts, particularly atomoxetine hydrochloride, have been employed as a pharmaceutically active agents in the treatment of attention deficit disorder (ADD) also known as attention deficit hyperactivity disorder (ADHD).

Based on short half-life of atomoxetine, initial atomoxetine trials involved twice-a-day dosing. Subsequently it was found that once-a-day dosing had comparable efficacy, suggesting that atomoxetine can be effectively dosed once-a-day. More recent studies in children have shown that therapeutic effect of atomoxetine diminishes after 12 hour post dose, even though benefits of single morning dose are detectable into the next day. The twice daily morning/early evening administration schedule is beneficial for many ADD patients because it provides desired plasma levels of atomoxetine to the patient for at least about 12 hours, in particular, at times of the day when symptoms are likely to be the most problematic. Additionally, it has been recently reported that children on BID dosing exhibit less intense stomach aches, nausea, and Oppositional Defiant Disorder (ODD) compared to children on QD dosing due to reduction in peak blood levels in a BID dosing regimen comprising splitting a single dose into two doses Waxmonsky et al. (2011). “A Comparison of Atomoxetine Administered as Once versus Twice Daily Dosing on the School and Home Functioning of Children with Attention-Deficit/Hyperactivity Disorder,” Journal of Child and Adolescent Psychopharmacology, Vol 21 (1): 21-32.

Atomoxetine is currently formulated as atomoxetine hydrochloride immediate release capsules containing 10 mg, 18 mg, 25 mg, 40 mg, and 60 mg, 80 mg, and 100 mg of atomoxetine hydrochloride. Atomoxetine is administered as a single daily dose or as divided twice daily doses for morning and afternoon/early evening administration.

Notwithstanding, providing desired plasma levels of atomoxetine for up to about 16 hours, e.g., about 12 hours and reducing side effects associated with high peak plasma levels of the drug. BID dosing requires two administrations of atomoxetine. This is particularly problematic for juvenile patients, where the second administration generally needs to be performed at school. Extended release medications are preferred over short-acting medications by many families affected with ADHD and by prescribing physicians with expertise in treating ADHD. Pelham et al. (2001). “Once-a-day Concerta methylphenidate versus three-times daily methylphenidate in laboratory and natural settings. Pediatrics,” 107 E105. It has been shown that children treated with extended release ADHD medications are less likely to switch, discontinue, or have gaps in their treatment, than children prescribed with IR ADHD medications. Lage M, Hwang, P. (2004). “Effect of Methylphenidate formulation for attention deficit hyperactivity disorder on patterns and outcomes of treatment,” Journal of Child and Adolescent Psychopharmacology, 14:575-81.

Therefore, it is desirable to develop extended release atomoxetine compositions suitable for once-daily administration. There is a need for once-a-day extended release atomoxetine compositions that provide extended release of atomoxetine or a pharmaceutically acceptable salt thereof for up to about 16 hours post administration, as provided by BID dosing of IR compositions, while reducing initial burst release and peak-to-trough fluctuations of the drug, generally associated with BID dosing of IR atomoxetine compositions. It is particularly desirable to develop extended release atomoxetine compositions suitable for once-daily administration, wherein the compositions provide extended release of atomoxetine or a pharmaceutically acceptable salt thereof in a therapeutic range, while reducing initial burst release and dose related peak plasma levels/Cₘₐₓ of atomoxetine, providing lower Cₘₐₓ:Cₘᵢₙ ratio, and Fluctuation Index (FI), for improved tolerability and reduced adverse effects associated with high plasma concentrations (e.g., concentrations beyond therapeutic range) and high peak-to-trough fluctuations of the drug. Extended release atomoxetine compositions suitable for once-a-day administration that provide desired plasma profiles of atomoxetine within therapeutic range are thus desirable.

It is desirable to develop extended release atomoxetine compositions suitable for once-daily administration for improving adherence, reducing stigma (because child or young person does not need to take medication at school), reducing problems associated with storing and administering controlled drugs, and improving PK profiles by reducing peak plasma concentrations (Cₘₐₓ), and peak-to-trough fluctuations, as compared to immediate release atomoxetine compositions administered twice-a-day. Improved control of atomoxetine pharmacokinetic properties may be achieved with alternative formulations.

The present invention addresses these and other needs for improved atomoxetine compositions, particularly extended-release dosage forms suitable for once daily administration.

### 4. SUMMARY

In certain embodiments, the present disclosure provides for a pharmaceutical pellet composition comprising: a core comprising atomoxetine or a pharmaceutically acceptable salt thereof; and a functional coat covering at least a portion of the core. The functional coat comprises a water-insoluble polymer and a pore former. The composition releases less than or equal to 40% of the atomoxetine or the pharmaceutically acceptable salt thereof, based on the total weight of atomoxetine or the pharmaceutically acceptable salt thereof present in the composition, within 2 hours of coming in contact with 900 mL of a dissolution medium comprising 0.05 M pH 6.8 buffer, measured using USP Apparatus II (paddle) at 50 rpm and 37° C.

In certain embodiments, the present disclosure provides for a pharmaceutical pellet composition comprising: a core comprising atomoxetine or a pharmaceutically acceptable salt thereof; and a functional coat covering at least a portion of the core. The functional coat comprises a water-insoluble polymer and a pore former. The composition provides a lag time of at least about 30 minutes, during which the composition releases less than or equal to 20% of atomoxetine or a pharmaceutically acceptable salt thereof, measured using USP Apparatus 2 (Paddle), with sinkers, in 900 mL of a dissolution medium comprising 0.05 M of pH 6.8 buffer, at 37° C. and 50 rpm.

In certain embodiments, the atomoxetine or the pharmaceutically acceptable salt thereof is present in an amount of from about 20% w/w to about 80% w/w, based on the total weight of the composition.

In certain embodiments, the water-insoluble polymer and the pore former are present in a weight ratio of from about 50:50 to about 99:1. In certain embodiments, the core further comprises an organic acid selected from the group comprising tartaric acid, citric acid, fumaric acid, succinic acid, malic acid, maleic acid, itaconic acid, gylcolic acid, and combinations thereof.

In certain embodiments, the water-insoluble polymer in the functional coat is selected from the group consisting of ethyl cellulose (ETHOCEL™), cellulose acetate, cellulose acetate butyrate, cellulose diacetate, cellulose triacetate, cellulose propionate, wax, copolymer of ethyl acrylate-methyl methacrylate-trimethylammonioethyl methacrylate chloride (1:2:0.1) (EUDRAGIT® RS copolymer), copolymer of ethyl acrylate-methyl methacrylate-trimethylammonioethyl methacrylate chloride (1:2:0.2) (EUDRAGIT® RL copolymer), copolymer of ethyl acrylate-methyl methacrylate (EUDRAGIT℠ NE, NM copolymer), a polyvinyl acetate dispersion (KOLLICOAT® SR), or mixtures thereof.

In certain embodiments, the pore former is a water-soluble plasticizer selected from the group comprising glycerin, polyethylene glycol monomethyl ether, triethyl citrate, triacetin, polyethylene glycol, propylene glycol, sorbitol sorbitan solution, or mixtures thereof.

In certain embodiments, the water-insoluble polymer and the pore former are present in a weight ratio of from about 50:50 to about 99:1.

In certain embodiments, the composition provides extended release for at least about 8 hours.

In certain embodiments, the composition is suitable for once daily administration.

In certain embodiments, the core is a pellet, bead/seed, or a granule coated with a drug layer comprising atomoxetine or a pharmaceutically acceptable salt thereof.

In certain embodiments, the core is a pellet, bead/seed or a granule containing atomoxetine or a pharmaceutically acceptable salt thereof.

In certain embodiments, the pellet, bead/seed, or a granule is a nonpareil bead/seed, or a granule.

In certain embodiments, the present disclosure provides for a method for making a pharmaceutical pellet composition comprising:
a) coating a nonpareil seed/bead, or a granule, with a drug layer comprising atomoxetine or a pharmaceutically acceptable salt thereof to obtain a drug layered core;
b) optionally, coating the drug layered core with a seal coat comprising water-soluble polymer to obtain a seal coated core; and
c) coating the core from step a) or step b) with a functional coat/extended release coat comprising a water-insoluble polymer, and at least one pore former and/or a plasticizer.
wherein the composition releases less than or equal to 40% of the atomoxetine or the pharmaceutically acceptable salt thereof within 2 hours of coming in contact with 900 mL of a dissolution medium comprising 0.05 M pH 6.8 buffer, measured using USP Apparatus II (paddle) at 50 rpm and 37° C.

In certain embodiments, the present disclosure provides for a method for making a pharmaceutical pellet composition comprising:
a) making a core containing atomoxetine or a pharmaceutically acceptable salt thereof using extrusion-spheronization process to obtain a drug containing core;
b) optionally, coating the drug containing core with a seal coat comprising water-soluble polymer to obtain a seal coated core; and
c) coating the core from step a) or step b) with a functional coat/extended release coat comprising a water-insoluble polymer, and at least one pore former and/or plasticizer.

In certain embodiments, the atomoxetine or a pharmaceutically acceptable salt thereof is present in an amount of from about 20% w/w to about 80% w/w, based on the total weight of the composition.

In certain embodiments, the water-insoluble polymer and the pore former are present in a weight ratio of from about 50:50 to about 99:1,

In certain embodiments, the drug layer further comprises an organic acid selected from the group consisting of tartaric acid, citric acid, fumaric acid, succinic acid, malic acid, maleic acid, itaconic acid, gylcolic acid, and combinations thereof.

In certain embodiments, the water-insoluble polymer in the functional coat is selected from the group consisting of ethyl cellulose (ETHOCEL™), cellulose acetate, cellulose acetate butyrate, cellulose diacetate, cellulose triacetate, cellulose propionate, wax, copolymer of ethyl acrylate-methyl methacrylate-trimethylammonioethyl methacrylate chloride (1:2:0.1) (EUDRAGIT©RS copolymer), copolymer of ethyl acrylate-methyl methacrylate-trimethylammonioethyl methacrylate chloride (1:2:0.2) (EUDRAGIT® RL copolymer), copolymer of ethyl acrylate-methyl methacrylate (EUDRAGIT® NE, NM copolymer), a polyvinyl acetate dispersion (KOLLICOAT® SR), or mixtures thereof.

In certain embodiments, the pore former is a water-soluble plasticizer is selected from the group comprising glycerin, polyethylene glycol monomethyl ether, triethyl citrate, triacetin, polyethylene glycol, propylene glycol, sorbitol sorbitan solution, or mixtures thereof.

In certain embodiments, the composition provides extended release for at least about 8 hours.

In certain embodiments, the present disclosure provides for a method for treating attention deficit hyperactivity disorder in a subject, the method comprising administering to the subject in need thereof, a pharmaceutical pellet composition comprising:
a) a core comprising atomoxetine or a pharmaceutically acceptable salt thereof; and
b) a functional coat covering at least a portion of the core. The functional coat comprises a water-insoluble polymer and a pore former. The composition releases less than or equal to 40% of the atomoxetine or the pharmaceutically acceptable salt thereof within 2 hours of coming in contact with 900 mL of a dissolution medium comprising 0.05 M pH 6.8 buffer, measured using USP Apparatus II (paddle) at 50 rpm and 37° C.

In certain embodiments, the composition provides extended release for at least about 8 hours.

In certain embodiments, the water-insoluble polymer and the pore former are present in a weight ratio of from about 50:50 to about 99:1

In certain embodiments, the core is a pellet, bead/seed, or a granule coated with a drug layer comprising atomoxetine or a pharmaceutically acceptable salt thereof.

In certain embodiments, the core is a pellet, bead/seed or a granule containing atomoxetine or a pharmaceutically acceptable salt thereof.

In certain embodiments, the pore former is a water-soluble plasticizer selected from the group comprising glycerin, polyethylene glycol monomethyl ether, triethyl citrate, triacetin, polyethylene glycol, propylene glycol, sorbitol, sorbitan solution, or mixtures thereof.

### 5. BRIEF DESCRIPTION OF THE DRAWINGS

FIG. 1 compares in vitro dissolution profiles of ATO HCl Pellet 1 (without acidifier) in 0.1 N HCl, pH 4.5 acetate buffer, pH 6.8 phosphate buffer, and pH 7.5 phosphate buffer dissolution mediums; with Strattera (60 mg) in 0.1N HCl dissolution medium. Dissolution studies were performed using USP Apparatus 2 (Paddle), with sinkers, in 900 mL of the dissolution medium. FIG. 1 shows that Pellet 1 provided extended release profile with a lag time of at least about 30 minutes and substantially reduced initial burst release of ATO HCl compared to Strattera.

FIG. 2 compares in vitro dissolution profiles of ATO HCl Pellet 2 (with acidifier) in 0.1 N HCl, pH 4.5 acetate buffer, pH 6.8 phosphate buffer, and pH 7.5 phosphate buffer dissolution mediums; with Strattera (60 mg) in 0.1N HCl dissolution medium. Dissolution studies were performed using USP Apparatus 2 (Paddle), with sinkers, in 900 mL of the dissolution medium. FIG. 2 shows that Pellet 2 provided extended release profile with a lag time of at least about 30 minutes and substantially reduced initial burst release of ATO HCl compared to Strattera.

FIG. 3 compares in vitro dissolution profiles of compositions comprising Pellet 11a with 6% functional coating weight gain, and composition comprising Pellet 11.b with 8% functional coating weight gain. Pellets 11.a and 11.b contained functional coat comprising ethyl cellulose and dibutyl sebacate in a wt. ratio of about 91:9. Dissolution tests were performed using USP Apparatus 2 (Paddle), with sinkers, in 900 mL of a dissolution medium comprising 0.05 M of pH 6.8 buffer, at 37° C. and 50 rpm. FIG. 3 shows a lag time of about 4 hours and drug recovery of about 29% w/w at 24 hours for Pellet 11.a with 6% w/w coating weight gain, and 20% drug release at 24 hours for Pellet 11.b with 8% w/w coating weight gain.

FIG. 4 compares in vitro dissolution profiles of compositions comprising Pellet 12. a with 6% w/w functional coating weight gain, and composition comprising Pellet 12.b with 8% w/w functional coating weight gain. Pellets 12.a and 12.b contained functional coat comprising cellulose acetate butyrate (CAB 171-15 NF), polyethylene glycol (PEG-3350) and hydroxypropyl methylcellulose phthalate (HPMC-P) in a wt. ratio of about 5:1:5. The dissolutions were performed using USP Apparatus 2 (Paddle), with sinkers, in three stage dissolution media comprising 500 mL of 0.01N HCl (0-1 hr); 750 mL of pH 4.5 acetate buffer (1-2 hrs); and then in 1000 mL of pH 6.8 buffer (2-24 hrs), at 37° C. and 50 rpm. FIG. 4 shows a lag time from the pellets for first 90 minutes followed by complete drug release/drug recovery by 4 hours.

FIG. 5 compares in vitro dissolution profiles of compositions comprising Pellet 13.a with 6% w/w functional coating weight gain, and composition comprising Pellet 13.b with 8% w/w functional coating weight gain. Pellets 13.a and 13.b contained functional coat comprising cellulose acetate butyrate (CAB 171-15 NF), polyethylene glycol (PEG-3350) and hydroxypropyl methylcellulose (HPMC) in a wt. ratio of about 10:1:1. The dissolutions were performed using USP Apparatus 2 (Paddle), with sinkers, in 900 mL of a dissolution medium comprising 0.05 M of pH 6.8 buffer, at 37° C. and 50 rpm. FIG. 5 shows a lag time of at least about 30 minutes followed by gradual release extending up to 12 hours for Pellet 13.a with 6% w/w coating weight gain and up to 20 hours for Pellet 13.b with 10% w/w coating weight gain. The compositions provide at least about 80% drug recovery from about 12 hours to about 20 hours.

FIG. 6 compares in vitro dissolution profiles of compositions comprising Pellet 14.a with 6% w/w functional coating weight gain, composition comprising Pellet 14b with 8% w/w functional coating weight gain, and composition comprising Pellet 14.c with 18% w/w functional coating weight gain. Pellets 14.a, 14.b, and 14.c contained functional coat comprising cellulose acetate butyrate (CAB 171-15 NF) and polyethylene glycol (PEG-3350) in a wt. ratio of about 5:1. The dissolutions were performed using USP Apparatus 2 (Paddle), with sinkers, in 900 mL of a dissolution medium comprising 0.05 M of pH 6.8 buffer, at 37° C. and 50 rpm. FIG. 6 shows a lag time of at least about 30 minutes, followed by extended release for about 12 hours for pellets with 6% w/w coating weight gain, about 16 hours for pellets with about 8% w/w coating weight gain, and for about 20 hours for pellets with 18% coating weight gain. The compositions provide at least about 80% drug recovery from about 12 hours to about 16 hours.

### 6. DETAILED DESCRIPTION

The presently disclosed subject matter provides ER ATO compositions suitable for once-daily administration. The once-a-day compositions improve patient adherence, reduce stigma (because child or young person does not need to take medication at school), reduce problems associated with storing and administering controlled drugs, and improve PK profiles by reducing peak plasma concentrations (Cₘₐₓ), and peak-to-trough fluctuations, as compared to immediate release atomoxetine compositions administered once or twice-a-day.

The ER compositions of the disclosure reduce drug toxicity and drug-related side effects, compared to IR capsules, by reducing initial burst release, providing lower Cₘₐₓ:Cₘᵢₙ ratio and Fluctuation Index, and provide consistent plasma levels of the drug in therapeutic range throughout the day.

The compositions of the disclosure provide membrane-controlled extended release of ATO or a pharmaceutically acceptable salt thereof, wherein the membrane reduces initial burst release, and controls and extends the release of the drug for up to about 16 hours. In certain embodiments, the compositions of the disclosure provide pH independent drug release as the dosage form passes through the portions of the GI tract with a pH of less than 4.5 or greater than 4.5. The membrane-controlled extended release helps the dosage form to provide and maintain at least a minimum therapeutic plasma concentration, without increased plasma levels associated with burst release of ATO or a pharmaceutically acceptable salt thereof. It is generally known that ATO exhibits highest solubility at pH of about 4.5 (about 52 mg/mL), with solubility decreasing approximately 3-fold at pH of less than 4.5 (e.g., about 18 mg/mL at pH of about 1) and decreasing approximately 10-fold at pH of greater than about 4.5 (about 5 mg/mL to about 7 mg/mL). Even though Atomoxetine Hydrochloride has demonstrated pH dependent solubility, it has been surprisingly found that the compositions exhibited pH independent drug release throughout the GI tract when coated with a water-insoluble polymer/functional coat. In addition, the compositions with cellulose acetate based polymers also minimized alcohol induced dose dumping of the drug. The ER atomoxetine compositions of the disclosure include pellets suitable for dosing in capsules, sachets, or sprinkling on food or liquid.

In certain embodiments, the compositions of the disclosure provide extended release of ATO or a pharmaceutically acceptable salt thereof up to about 16 hours, and exhibit a shelf life of at least about 2 years at controlled room temperature conditions.

In certain embodiments, the disclosure provides methods for making ER pellets of ATO. In certain embodiments, the disclosure provides methods for making ATO capsules containing extended release ATO pellets. In certain embodiments, the disclosure provides methods for improving patient compliance by administering to the patient an extended release ATO composition that can provide extended release profile with reduced dose related peak-to-trough fluctuations, e.g., providing lower Cₘₐₓ:Cₘᵢₙ ratio, for improved tolerability and reduced adverse effects associated with high plasma concentrations and high peak-to-trough fluctuations. In certain embodiments, the disclosure provides methods for improving patient compliance by administering to the patient an extended release atomoxetine composition that can be administered as pellets for dosing in capsules, sachets, and as sprinkles on food. In certain embodiments, the disclosure provides methods of treating Attention-Deficit/Hyperactivity Disorder (ADHD), using extended release atomoxetine pellets of the disclosure.

For clarity and not by way of limitation, this detailed description is divided into the following subportions:
6.1. Definitions;
6.2. Formulations of Pellet Dosage Forms;
6.3. Compositions;
6.4. Methods of Making; and
6.5. Methods of Use.

### 6.1. Definitions

The terminology used in the present disclosure is for the purpose of describing particular embodiments only and is not intended to be limiting. Certain terms are discussed below, or elsewhere in the specification, to provide additional guidance in describing the compositions and methods of the disclosure and how to make and use them. Unless otherwise defined, all terms, including technical and scientific terms used in the description, have the same meaning as commonly understood by one of ordinary skill in the art to which this disclosure belongs.

The singular forms “a,” “an,” and “the,” as used herein, include plural references unless the context dictates otherwise. Still further, the terms “having,” “including,” “containing,” and “comprising” are interchangeable, and one of skill in the art is cognizant that these terms are open-ended terms.

As used herein, the term “or” is a logical disjunction (i.e., and/or) and does not indicate an exclusive disjunction unless expressly indicated such as with the terms “either,” “unless,” “alternatively,” and words of similar effects.

The term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 15%, up to 10%, up to 5%, up to 1%, up to 0.5%, or even up to 0.1% of a given value.

As used herein, a “therapeutically effective,” “therapeutic,” or “therapeutically acceptable” amount refers to an amount that will elicit a therapeutically useful response in a subject and includes an additional amount or overage of active ingredient deemed necessary in the formulation to provide the desired amount upon administration. The therapeutically useful response can provide some alleviation, mitigation, and/or decrease in at least one clinical symptom in the subject. Those skilled in the art will appreciate that the therapeutically useful response need not be complete or curative, as long as some benefit is provided to the subject. In some embodiments, the subject is a human.

As used herein, the term “drug recovery” refers to percentage of the total amount of drug present in the dosage form that is released in a dissolution medium. The term “complete drug recovery” refers to release of about 90% to about 105% of the drug present in the dosage form.

As used herein, the term “burst release” refers to release of ATO or a salt thereof in an amount that is outside (i.e., above) the therapeutic range, and providing a drug plasma concentration level that can result in various unwanted side effects. The term “therapeutic range” includes a range of the amount of drug that will elicit a therapeutically useful response in a subject and includes an additional amount or overage of active ingredient deemed necessary in the formulation to provide the desired amount upon administration. Alternately, the term “burst release,” in in vitro settings, refers to release of ≥about 40% by wt. of ATO or a salt thereof in about 2 hours or less, in 900 mL of 0.05 M pH 6.8 buffer, measured using USP Apparatus II (paddle) at 50 rpm and 37° C.

As used herein, the term “lag time” refers to time to drug release from the compositions of the disclosure, measured from the time of contact of the composition with a dissolution medium. In certain embodiments, under in vitro settings, the “lag time” includes time to drug release in 900 mL of 0.05 M pH 6.8 buffer, measured using USP Apparatus II (paddle) at 50 rpm and 37°. In certain embodiments, the compositions of the disclosure provide a lag time of from about 30 minutes to about 240 minutes, during which the compositions release ≤about 20% by wt. of ATO or a salt thereof.

As used herein, the terms “drug layer,” refers to a coating layer comprising ATO or a pharmaceutically acceptable salt thereof.

As used herein, the term “coating weight gain” refers to coating weight gain with respect to the weight of the uncoated pellet. For example, a coating weight gain of 15% refers to a 15 wt % increase in pellet weight during coating with respect to the uncoated pellet weight.

As used interchangeably herein, the terms “modified release,” “extended release,” and “sustained release” refer to drug dosage forms or compositions that are formulated to make the drug available over an extended period after ingestion/consumption/administration, thereby allowing a reduction in dosing frequency, as compared to a drug presented as an immediate release dosage form (e.g., as a solution or an immediate release dosage form). In certain embodiments, the terms “extended release” and “sustained release” are used interchangeably to refer to dosage forms/compositions that are formulated to maintain relatively consistent drug concentrations in plasma during a dosing interval comprising an extended period of time (i.e., post-administration), as compared to the drug administered as an immediate release dosage form.

As used interchangeably herein, the terms “extended period of time” and “extended period” refer to a time period of up to about 16 hours.

As used herein, the term “release rate” refers to the quantity of drug released per unit time, e.g., mg of drug released per hour (mg/hour), from a dosage form. In certain embodiments, drug release rates can be calculated under in vitro dosage form dissolution testing conditions known in the art.

As used herein, the term “immediate release” refers to release of at least 80% of a drug in about one hour or less, preferably within 45 minutes or less, post-administration.

The terms “functional coat” and “extended release coating” are used interchangeably herein.

As used herein, the terms “pore former” and the like refer to pH-dependent or pH-independent water-soluble polymers and/or water-soluble small molecules that can form pores or channels (i.e., behave as a channeling agent) in the functional coat, thereby creating a permeable functional coat/membrane. The term “pore former” includes molecules used to create a certain amount of diffusion through the membrane/coating of a tablet, pellet, or particle to achieve a sustained/extended release profile. In certain embodiments, the pore former includes water-soluble polymers. In certain embodiments, the pore former includes enteric polymers. In certain embodiments, plasticizers are pore formers.

As used interchangeably herein, the terms “core”, “drug containing core” and “drug layered core” refer to pellet, beads/seed, or granule comprising ATO or a pharmaceutically acceptable salt thereof, and optionally, at least one organic acid; or an inert substrate/nonpareil seed coated with a coating/drug layer comprising ATO or a pharmaceutically acceptable salt thereof, and optionally, at least one organic acid. In certain embodiments, the core is an organic acid granule coated with a drug layer containing atomoxetine or a pharmaceutically acceptable salt thereof.

As used interchangeably herein, the terms “inert substrate,” and “nonpareil seed” refer to inert material/particles, e.g., pellets, beads, seeds, or granules. In certain embodiments, the nonpareil seeds are composed of inert substrates such as starch, sugar, lactose, and/or cellulose. In certain embodiments, the nonpareil seed is sugar sphere or microcrystalline cellulose.

As used herein, the term “gastric fluid” refers to medium occurring in the stomach and upper GI tract of an individual.

As used herein, the term “simulated gastric fluid” refers to a medium that is used to mimic the chemical environment of gastric fluid/medium in an in vitro setting.

As used herein, the term “medium” refers to a dissolution medium used to mimic pH of GI tract of an individual. In certain embodiments, the medium used to mimic the environment of stomach of an individual includes a medium with pH of less than about 5.5. In certain embodiments, the medium used to mimic the environment of lower GI tract of an individual includes a medium with pH of from about 5.5 to about 8.

As used herein, the term “bioavailability” refers to the fraction of an administered dose of unchanged drug that reaches the systemic circulation.

As used herein, the term “substantially free” means that the composition comprises less than 0.001 wt % of the material.

As used herein, the term “microenvironment” refers to immediate environment of ATO within the pellet.

As used herein, the term “acid microenvironment” refers to a microenvironment of ATO with pH of less than about 5.

As used herein, the term “therapeutic concentration” refers to a plasma concentration of ATO or pharmaceutically acceptable salts thereof providing therapeutically useful response.

The terms “Fluctuation Index,” and “FI,” as used interchangeably herein with respect to ATO compositions, refer to fluctuations in plasma levels of ATO or a pharmaceutically acceptable salt thereof released from the composition during a 24-hour dosing period. FI provides a quantitative measurement of fluctuation in drug plasma concentration, measured as dose-related peak-to-trough fluctuations. FI is calculated using the formula: FI=(Cₘₐₓ−Cₘᵢₙ)/Cₐᵥ.

Cₘₐₓ is maximum (or peak) concentration of ATO or a pharmaceutically acceptable salt thereof in the plasma after the drug has been administered during the time interval of interest.

Cₘᵢₙ is minimum concentration of ATO or a pharmaceutically acceptable salt thereof in the plasma after the drug has been administered during the time interval of interest. This definition is slightly different from C_{trough}, the concentration immediately prior to administration of the next dose.

The term “Cₐᵥ” as used herein with respect to ATO compositions, refers to average plasma concentration of ATO or pharmaceutically acceptable salt thereof, during a 24 hour dosing period.

The term “alcohol induced dose dumping,” as used herein, refers to rapid unintended release of a large amount of drug from a modified release/extended release/sustained release dosage form resulting from an accidental misuse or from intentional abuse of alcohol with the drug.

As used herein, the terms “treatment,” “treat,” and “treating” refer to reversing, alleviating, delaying the onset of, and/or inhibiting the progress of a disease or disorder as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

As used interchangeably herein, the terms “atomoxetine,” and “ATO,” include all pharmaceutically acceptable salts, esters, and functionally equivalent chemical compounds, including Atomoxetine hydrochloride. “Atomoxetine” and “ATO” refer to the compound with the IUPAC name (−)-N-methyl-3-phenyl-3-fo-tolyloxy)propyl amine, molecular formula C17H21NO, having the structure:

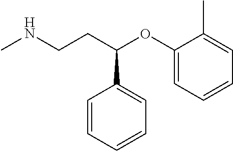 [SMILES: CNCC[C@@H](Oc1ccccc1C)c1ccccc1]

As used herein, the terms “up,” “down,” “above,” “below,” “top,” “bottom,” etc. should be interpreted as nonlimiting upon the pellets, cores, layers, methods, and products of any methods of present disclosure, which can be spatially arranged in any orientation or manner.

### 6.2. Formulations of Pellet Dosage Forms

The present disclosure provides extended release oral ATO compositions that maintain solubility of the drug in different pH environments of the GI tract and maintain a therapeutic plasma concentration of the drug for extended periods of time, without any spike or burst in release of the drug.

ATO is commonly prescribed as ATO HCl. However, use of other pharmaceutically acceptable salts of ATO is also contemplated in the present disclosure.

Pharmaceutically acceptable salts of ATO known in the art include, but are not limited to, hydrochloride, hydrobromide, hydroiodide, bromide, sulfite, sulfate, bisulfate, nitrate, salicylate, citrate, tartrate, bitartrate, lactate, phosphate, malate, maleate, fumarate, succinate, acetate, and pamoate salts.

In certain embodiments, compositions of the present disclosure comprise ATO extended release pellets. In certain embodiments, the compositions described herein comprise capsules containing extended release pellets. In certain embodiments, the extended release pellets comprise a core comprising a nonpareil pellet, bead/seed, drug containing core, or a granule coated/layered with a drug layer comprising ATO or a pharmaceutically acceptable salt thereof, and optionally, at least one organic acid. In certain embodiments, the extended release pellets comprise a core that is a pellet, bead/seed, drug containing core, or a granule comprising ATO and, optionally, at least one organic acid. In certain embodiments, the core is coated with a seal coat, and a functional coat/membrane/extended release coat over the core. In certain embodiments, the presence of seal coat is optional. Each component of the compositions of the present disclosure is described in more detail below.

### 6.2.1. Drug Containing Core

In certain embodiments, the present disclosure provides pellets comprising a core/drug layered core that comprises ATO or a pharmaceutically acceptable salt thereof and, optionally, at least one organic acid. In certain embodiments, the presence of organic acid in the core provides an acid microenvironment around ATO or a pharmaceutically acceptable salt thereof to increase solubility of ATO at pH of greater than or equal to 4.5. In certain embodiments, the compositions of the present disclosure provide extended release of ATO or a pharmaceutically acceptable salt thereof for up to 16 hours, e.g., about 12 hours.

In certain embodiments, weight ratio atomoxetine or a pharmaceutically acceptable salt thereof and organic acid is from about 50:50 to about 100:0. In certain embodiments, the ratio of atomoxetine hydrochloride to organic acid is about 50:50, about 55:45, about 60:40, about 65:35, about 70:30, about 75:25, about 80:20, about 85:15, about 90:10, about 95:5, about 100:0, or any intermediate values. In certain embodiments, the core/drug layered core makes up from about 40% to about 99%, from about 50% to about 95%, from about 60% to about 90% w/w, or any intermediate values thereof, of the total weight of the composition of the present disclosure. In certain embodiments, the core makes up at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, about 91%, about 92%, about 93%, about 94%, about 95%, about 96%, about 97%, about 98% w/w, or intermediate values therein, of the total weight of the composition of the present disclosure.

### Pellets, Beads/Seeds, or Granules Coated with a Drug Layer Containing ATO or a Pharmaceutically Acceptable Salt Thereof

In certain embodiments, the core is spherical or irregular in shape. In certain embodiments, the core is a nonpareil seed coated with a drug layer comprising ATO or a pharmaceutically acceptable salt thereof, and, optionally, at least one organic acid, e.g., a cellets, suglets/sugar sphere coated with a drug layer. In certain embodiments, the core is a microcrystalline cellulose sphere/cellet or a sugar sphere coated with a drug layer comprising ATO or a pharmaceutically acceptable salt thereof and, optionally, at least one organic acid. In certain embodiments, the core is a nonpareil seed/bead or a granule coated with a coating comprising at least one organic acid to provide an acid coated core that is further coated with a drug layer comprising ATO or a pharmaceutically acceptable salt thereof. In certain embodiments, the drug layer on the acid coated core can further comprises at least one organic acid. In certain embodiments, the acid coated core and the drug layer contain same organic acid. In certain embodiments, the acid coated core and the drug layer contain different organic acids. In certain embodiments, the core comprises an acid pellet, bead/seed, or a granule coated with a drug layer. In certain embodiments, the drug layer comprises ATO or a pharmaceutically acceptable salt thereof and, optionally, at least one organic acid. In certain embodiments, the acid in acid pellet, bead/seed, or granule and in the drug layer is same. In certain embodiments, the acid in acid pellet, bead/seed, or granule and in the drug layer is different.

In certain embodiments, the core consists of one or more organic acids. In certain embodiments, the organic acid in the core is present in an amount of from about 0% to about 100%, from about 20% to about 80%, from about 30% to about 70% w/w, or any intermediate values thereof, based on the total weight of the core. In certain embodiments, the organic acid is present in amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% w/w, based on the total weight of the core.

In certain embodiments, the organic acid is selected from the group consisting of tartaric acid, citric acid, fumaric acid, succinic acid, malic acid, maleic acid, glycolic acid, itaconic acid or any combinations thereof. In certain embodiments, the organic acid is tartaric acid or fumaric acid.

In certain embodiments, the core is a nonpareil seed, e.g., microcrystalline cellulose sphere or a sugar sphere coated with a drug layer comprising ATO or a pharmaceutically acceptable salt thereof and, optionally, at least one organic acid. In certain embodiments, the drug layer covers at least a portion of the nonpareil seed. In certain embodiments, the drug layer comprises ATO or a pharmaceutically acceptable salt thereof and a water-soluble polymer. In certain embodiments, the drug layer further comprises at least one organic acid. In certain embodiments, the organic acid includes, but is not limited to, tartaric acid, citric acid, fumaric acid, succinic acid, malic acid, maleic acid and combinations thereof. In certain embodiments, coating solvents used for coating the drug layer comprise, but are not limited to, an organic solvent, water, and/or any mixtures thereof. In certain embodiments, the coating solvent is a mixture of an organic solvent and water. In certain embodiments, organic solvent:water weight ratio is between 60:40 and 100:0. In certain embodiments, the organic solvent:water weight ratio is about 60:40, about 70:30, about 75:25, 80:20, about 85:15, about 90:10, about 95:5, about 98:2, or any intermediate values therein. In certain embodiments, the coating solvents comprise organic solvents selected from the group consisting of methylene chloride, carbon tetrachloride, acetone, methanol, ethanol, isopropyl alcohol, and/or any mixtures thereof. In certain embodiments, coating solvents comprise, but are not limited to, methylene chloride, carbon tetrachloride, acetone, methanol, ethanol, water, and/or any mixtures thereof. In certain embodiments, the coating solvent is a mixture of methanol and water. In certain embodiments, methanol:water weight ratio is between 60:40 and 98:2. In certain embodiments, the methanol:water weight ratio is about 60:40, about 70:30, about 75:25, 80:20, about 85:15, about 90:10, about 95:5, about 98:2, or any intermediate values therein.

In certain embodiments, the ATO or a pharmaceutically acceptable salt thereof is present in a concentration of from about 10% to about 99%, from about 15% to about 90%, from about 20% to about 85%, from about 25% to about 80%, or from about 30% to about 75% w/w, based on the total weight of the drug layer composition. In certain embodiments, ATO or a pharmaceutically acceptable salt thereof is present in an amount of about 40% to about 70%, about 45%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70% w/w, or any intermediate values therein, based on the total weight of the drug layer composition. In certain embodiments, ATO is present in a concentration of from about 30% w/w to about 90% w/w, based on the total weight of the composition. In certain embodiments, ATO or a pharmaceutically acceptable salt thereof is present in an amount of about 40% to about 70% w/w, about 45%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, or any intermediate values therein, based on the total weight of the composition.

In certain embodiments, the drug layer comprises a water-soluble polymer. In certain embodiments, the water-soluble polymer in the drug layer is selected from the group consisting of hydroxypropyl cellulose, methyl cellulose, hydroxypropyl methylcellulose, hydroxyethyl cellulose, polyethylene glycol, polyvinyl alcohol, polyvinyl pyrrolidine (PVP), polyvinyl pyrrolidone-vinyl acetate copolymer (Copovidone), poloxamer, and mixtures thereof. In certain embodiments, the amount of the water-soluble polymer ranges from about 1% to about 90% w/w, based on the total weight of the drug layer composition. In certain embodiments, the concentration of the water-soluble polymer ranges from about 1% to about 90%, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90% w/w, or intermediate values therein, based on the total weight of the drug layer composition.

In certain embodiments, the drug layer further includes additional excipients comprising anti-tacking agents, and/or plasticizers. In certain embodiments, anti-tacking agents include, but are not limited to, silicon dioxide (SYLOID® 244FP), fumed silica (CAB-O-SIL®), talc, kaolin, magnesium trisilicate, powdered starch, glyceryl monostearate, and/or tribasic calcium phosphate. In certain embodiments, the anti-tacking agent can be present in an amount of about 0% to about 80% w/w, based on the total weight of the water-soluble polymer present in the drug layer composition. In certain embodiments, the anti-tacking agent is present in an amount of about 10% w/w, about 15%, about 20%, about 25%, about 30%, about 35, about 40%, about 45%, about 50%, about 55%, about 60%, about 65%, about 70%, about 75%, about 80% w/w, or intermediate values therein, based on the total weight of the water-soluble polymer present in the drug layer composition.

In certain embodiments, the plasticizers include, but are not limited to, glycerin, triethyl citrate, triacetin, polyethylene glycol, propylene glycol, sorbitol, sorbitan solution, and/or dibutyl sebacate. In certain embodiments, the plasticizer is triethyl citrate. In certain embodiments, the plasticizer is dibutyl sebacate. In certain embodiments, the plasticizer is present in an amount of about 0%, about 0.1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% w/w, or any intermediate values therein, based on the total weight of water-soluble polymer present in the drug layer composition.

In certain embodiments, the drug layer comprises at least one acidifier that is an organic acid. In certain embodiments, the acidifier in drug layer is selected from the group consisting of tartaric acid, citric acid, fumaric acid, succinic acid, malic acid, maleic acid or any combinations thereof. In certain embodiments, the acidifier is tartaric acid.

### Pellets, Beads/Seeds, or Granules Containing ATO or a Pharmaceutically Acceptable Salt Thereof

In certain embodiments, the core comprises pellets, beads/seeds, or granules comprising ATO or a pharmaceutically acceptable salt thereof and a binder. In certain embodiments, the core further comprises at last one organic acid and/or at least one filler/bulking agent.

In certain embodiments, ATO or a pharmaceutically salt thereof is present in a concentration of from about 30% w/w to about 99% w/w, based on the total weight of the composition. In certain embodiments, ATO or a pharmaceutically acceptable salt thereof is present in an amount of about 40% to about 70%, about 45%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70% w/w, or any intermediate values therein, based on the total weight of the composition.

In certain embodiments, the binder is selected from the group consisting of hydroxypropyl cellulose, methyl cellulose, sodium methyl cellulose, starch, acacia, gellan gum, tragacanth, hydroxypropyl methylcellulose, hydroxyethyl cellulose, polyethylene glycol, polyvinyl alcohol, polyvinyl acetates, alginates, pegylated polyvinyl alcohol, polyvinyl pyrrolidone, and mixtures thereof. In certain embodiments, binder is a low viscosity hydroxypropyl cellulose. In certain embodiments, the amount of the binder ranges from about 0.5% to about 20% w/w, based on the total weight of the core. In certain embodiments, the concentration of the binder is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% w/w, or intermediate values therein, based on the total weight of the core.

In certain embodiments, the core further comprises at least one organic acid/acidifier to provide acid microenvironment for improving solubility of ATO or a pharmaceutically acceptable salt thereof in the GI tract. In certain embodiments, the organic acid is selected from the group comprising, but is not limited to, tartaric acid, citric acid, fumaric acid, succinic acid, malic acid, maleic acid and combinations thereof. In certain embodiments, the acidifier is present in an amount of from about 5% w/w to about 50% w/w, based on the total weight of the core. In certain embodiments, acid is present in an amount of about 5%, about 10%, about 15%, about 20%, about 25%, about 30t %, about 35%, about 40%, about 45%, about 50%, or any intermediate values therein, based on the total weight of the core.

In certain embodiments, the core comprises microcrystalline cellulose as a bulking agent.

### 6.2.2. Seal Coat and Over Coat

In certain embodiments, the pellets include at least one seal coat. In certain embodiments, the drug containing core is coated with a seal coat. In certain embodiments, the seal coat is optional. In certain embodiments, the pellets include a seal coat between the pellet core (e.g., core containing ATO or a pharmaceutically acceptable salt thereof or a core coated with a drug layer containing ATO or a pharmaceutically acceptable salt thereof) and a functional coat/extended release coat. In certain embodiments, drug layered pellets include two seal coats (seal coat-1 between the core and drug layer, and the seal coat-2 between the drug layer and functional coat/extended release coat). In certain embodiments, the pellets include an over coat. In certain embodiments, the presence of seal coat and/or the overcoat is optional. In certain embodiments, pellets with a core containing ATO or a pharmaceutically acceptable salt thereof do not contain a seal coat between the pellet core and functional coat/extended release coat. In certain embodiments, the pellets include various components and coats in the following order: a core (containing drug or coated with a drug layer); seal coat over the core and covering at least a portion of the core; and a functional coat/extended release coat over the seal coat and covering at least a portion of the seal coat. In certain embodiments, drug layered pellets include seal coat (e.g., seal coat-1) between the nonpareil seed and the drug layer, and covering at least a portion of the nonpareil seed. In certain embodiments, the seal coat(s), and/or the over coat are present at a coating weight gain of between 0% and about 20% w/w, based on the total weight of the corresponding pellet without seal coat, and/or the over coat. In certain embodiments, the seal coat and/or the over coat are present at a coating weight gain of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or any intermediate values therein, based on the total weight of the corresponding pellet without seal coat and/or the over coat. In certain embodiments, coating solvents used for coating the seal coat and/or the over coat comprise, but are not limited to, an organic solvent, water, and/or any mixtures thereof. In certain embodiments, the coating solvent is a mixture of an organic solvent and water. In certain embodiments, organic solvent:water weight ratio is between 60:40 and 100:0. In certain embodiments, the organic solvent:water weight ratio is about 60:40, about 70:30, about 75:25, 80:20, about 85:15, about 90:10, about 95:5, about 98:2, or any intermediate values therein. In certain embodiments, the coating solvents comprise organic solvents selected from the group consisting of methylene chloride, carbon tetrachloride, acetone, methanol, ethanol, isopropyl alcohol, and/or any mixtures thereof. In certain embodiments, coating solvents comprise, but are not limited to, methylene chloride, carbon tetrachloride, acetone, methanol, ethanol, water, and/or any mixtures thereof. In certain embodiments, the coating solvent is a mixture of ethanol and water. In certain embodiments, ethanol:water weight ratio is from about 60:40 to about 98:2. In certain embodiments, the methanol:water weight ratio is about 60:40, about 70:30, about 75:25, 80:20, about 85:15, about 90:10, about 95:5, about 98:2, or any intermediate values therein.

In certain embodiments, the seal coat(s) and over coat comprise a water-soluble polymer. In certain embodiments, the water-soluble polymer is selected from a group comprising hydroxypropyl cellulose, methyl cellulose, hydroxypropyl methylcellulose, hydroxyethyl cellulose, polyethylene glycol, polyvinyl alcohol, poloxamer, and mixtures thereof. In certain embodiments, the amount of the water-soluble polymer ranges from about 5% to about 100% w/w, based on the total weight of the seal coat/over coat composition. In certain embodiments, the amount of the water-soluble polymer ranges from about 2% to about 99%, from about 5% to about 95%, from about 15% to about 90%, from about 20% to about 85%, from about 25% to about 80%, from about 30% to about 75%, from about 35% to about 70%, from about 40% to about 65%, from about 45% to about 60%, or from about 50% to about 55% w/w, based on the total weight of the seal coat/over coat composition. In certain embodiments, the water-soluble polymer is present in an amount of about 2%, about 5%, about 7.5%, about 10%, about 12.5%, about 15%, about 17.5%, about 20%, about 22.5%, about 25%, about 27.5%, about 30%, about 32.5%, about 35% w/w, or any intermediate values therein, based on the total weight of the seal coat/over coat.

In certain embodiments, the composition of the seal coat/over coat further comprises additional excipients, such as anti-tacking agents and/or plasticizers. In certain embodiments, anti-tacking agents include, but are not limited to, silicon dioxide (SYLOID® 244FP), fumed silica (CAB-O-SIL®), talc, kaolin, magnesium trisilicate, powdered starch, tribasic calcium phosphate, glyceryl monostearate, and any combinations thereof. In certain embodiments, the anti-tacking agent can be present in an amount of from about 5% to about 85% w/w, based on the total weight of the seal coat/over coat composition. In certain embodiments, the anti-tacking agent is present in an amount of about 25%, about 30%, about 35%, about 40%, about 45%, about 50%, about 55%, about 60%, about 61t %, about 62%, about 63%, about 64%, about 65%, about 66%, about 67%, about 68%, about 69%, about 70%, about 71%, about 72%, about 73%, about 74%, about 75%, about 76%, about 77%, about 79%, about 80%, about 85% w/w, or any intermediate values therein, based on the total weight of the seal coat/overcoat composition.

### 6.2.3. Functional Coat/Extended Release Layer/Membrane

In certain embodiments, the pellet cores (with or without a seal coat) are further coated with a functional coat comprising a water-insoluble polymer, and a pore former. In certain embodiments, functional coat further comprises a plasticizer. In certain embodiments, plasticizer acts as a pore former as well. In certain embodiments, the functional coat covers at least a portion of the drug layer. In certain embodiments, there is a seal coat between the drug layer and the functional coat. In certain embodiments, the pellets comprising a core that is a drug layered core or a core containing the drug within the core, contain a seal coat between the core and functional coat. In certain embodiments, coating solvents used for coating the functional coat comprise, but are not limited to, an organic solvent, water, and/or any mixtures thereof. In certain embodiments, the coating solvent is a mixture of an organic solvent and water. In certain embodiments, organic solvent:water weight ratio is between 60:40 and 100:0. In certain embodiments, the organic solvent:water weight ratio is about 60:40, about 70:30, about 75:25, 80:20, about 85:15, about 90:10, about 95:5, about 100:0, or any intermediate values therein. In certain embodiments, the coating solvents comprise organic solvents selected from the group consisting of methylene chloride, carbon tetrachloride, acetone, methanol, ethanol, isopropyl alcohol, and/or any mixtures thereof. In certain embodiments, coating solvents comprise, but are not limited to, methylene chloride, carbon tetrachloride, acetone, methanol, ethanol, isopropyl alcohol, water, and/or any mixtures thereof. In certain embodiments, the coating solvent is a mixture of acetone and water. In certain embodiments, acetone:water weight ratio is between 60:40 and 98:2. In certain embodiments, the acetone:water weight ratio is about 60:40, about 70:30, about 75:25, 80:20, about 85:15, about 90:10, about 95:5, about 100:0, or any intermediate values therein.

In certain embodiments, the functional coat has a coating weight gain of about 1% to about 50% w/w, based on the total weight of the pellet without functional coat. In certain embodiments, the functional coat has a coating weight gain of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50% w/w, or any intermediate values therein, based on the total weight of the pellet without the functional coat.

In certain embodiments, the functional coat comprises a water-insoluble polymer, and at least one additional excipient comprising a pore former. In certain embodiments the functional coat further comprises a plasticizer. In certain embodiments, pore former is a water-soluble plasticizer. In certain embodiments, the pore former is a pH independent water-soluble polymer. In certain embodiments, the pore former is an enteric polymer. In certain embodiments, plasticizers include, but are not limited to, glycerin, polyethylene glycol monomethyl ether, triethyl citrate, triacetin, polyethylene glycol, propylene glycol, sorbitol sorbitan solution, dibutyl sebacate, or mixtures thereof. In certain embodiments, the plasticizer is triethyl citrate. In certain embodiments, the plasticizer is polyethylene glycol (e.g., PEG 3350). In certain embodiments, the plasticizer is present in an amount of about 0.1%, about 1%, about 2%, about 3%, about 4%, about 5%, about 6%, about 7%, about 8%, about 9%, about 10%, about 110, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50% w/w, or any intermediate values therein, based on the total weight of the functional coat.

In certain embodiments, the enteric polymer used as a pore former comprises, but is not limited to, cellulose acetate phthalate, cellulose acetate succinate, methylcellulose phthalate, hydroxyethyl cellulose phthalate, polyvinyl acetate phthalate, polyvinyl butyrate acetate, vinyl acetate-maleic anhydride copolymer, hydroxypropyl methylcellulose phthalate, polyvinyl acetate phthalate, cellulose acetate phthalate, methacrylic acid and methyl methacrylate (1:2) copolymer (EUDRAGIT© S 100), methacrylic acid and methyl methacrylate (1:1) copolymer (EUDRAGIT® L 100), methacrylic acid and methyl methacrylate (1:2) copolymer solution (EUDRAGIT® S 12.5), methacrylic acid and methyl methacrylate (1:1) copolymer solution (EUDRAGIT® L 12.5), methacrylic acid and ethyl acrylate copolymer/copolymer dispersion (EUDRAGIT® L 100-55), and combinations thereof. In certain embodiments, the enteric polymer is present in an amount of from about 0.10% to about 60% w/w, based on the total weight of the functional coat. In certain embodiments, the enteric polymer is present in an amount of about 0.1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60% w/w, or any intermediate values therein, based on the total weight of the functional coat.

In certain embodiments, the pore former is a water-soluble polymer comprising, but not limited to, methyl cellulose, hydroxypropyl cellulose, hydroxypropyl methylcellulose, hydroxyethyl cellulose, polyethylene glycol, polyvinyl alcohol, poloxamer, and mixtures thereof. In certain embodiments, the water-soluble polymer is present in an amount of from about 0.10% to about 60% w/w, based on the total weight of the functional coat. In In certain embodiments, the water-soluble polymer is present in an amount of about 00.1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60% w/w, or any intermediate values therein, based on the total weight of the functional coat.

In certain embodiments, the water-insoluble polymer in the functional coat comprises, but is not limited to, ethyl cellulose (ETHOCEL™), cellulose acetate, cellulose acetate butyrate, cellulose diacetate, cellulose triacetate, cellulose propionate, wax, copolymer of ethyl acrylate-methyl methacrylate-trimethylammonioethyl methacrylate chloride (1:2:0.1) (EUDRAGIT® RS copolymer), copolymer of ethyl acrylate-methyl methacrylate-trimethylammonioethyl methacrylate chloride (1:2:0.2) (EUDRAGIT® RL copolymer), copolymer of ethyl acrylate-methyl methacrylate (EUDRAGIT® NE, NM copolymer), a polyvinyl acetate dispersion (KOLLICOAT® SR), or mixtures thereof. In certain embodiments, presence of cellulose acetate based polymers as water-insoluble polymers reduces alcohol induced dose dumping of the atomoxetine or a pharmaceutically acceptable salt thereof. In certain embodiments, presence of cellulose acetate butyrate reduces alcohol induced dose dumping of atomoxetine or a pharmaceutically acceptable salt thereof. In certain embodiments, presence of cellulose acetate based polymers as water-insoluble polymers reduces initial burst release of atomoxetine or a pharmaceutically acceptable salt thereof, wherein the“initial burst release” is release of ≥about 40% by wt. of ATO or a salt thereof in about 2 hours or less, in 900 mL of 0.05 M pH 6.8 buffer, measured using USP Apparatus II (paddle) at 50 rpm and 37° C.

In certain embodiments, compositions of the disclosure comprising cellulose acetate based polymers as water-insoluble polymers provide a lag time of from about 30 minutes to about 240 minutes, measured in in vitro settings comprising 900 mL of 0.05 M pH 6.8 buffer, using USP Apparatus II (paddle) at 50 rpm and 37° C. In certain embodiments, compositions of the disclosure comprising cellulose acetate based polymers as water-insoluble polymers provide a lag time of at least about 30 minutes. In certain embodiments, compositions of the disclosure comprising cellulose acetate based polymers as water-insoluble polymers provide a lag time of at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, at least about 120 minutes, at least about 130 minutes, at least about 140 minutes, at least about 150 minutes at least about 160 minutes, at least about 170 minutes, at least about 180 minutes, at least about 190 minutes, at least about 200 minutes, at least about 210 minutes, at least about 220 minutes, at least about 230 minutes, at least about 240 minutes, or intermediate values thereof. In certain embodiments, the “lag time,” is the time during which the compositions of the disclosure release ≤about 20% by wt. of ATO or a salt thereof, based from the time of administration of the compositions, as measured in in vitro settings, in 900 mL of 0.05 M pH 6.8 buffer, measured using USP Apparatus II (paddle) at 50 rpm and 37° C.

In certain embodiments, compositions of the disclosure comprising cellulose acetate based polymers as water-insoluble polymers provide a lag time to drug release of at least about 30 minutes and provide a complete drug recovery from about 12 hours to about 24 hours. In certain embodiments, the complete drug recovery comprises release of more than 80% by wt of the drug, e.g., ATO or a pharmaceutically acceptable salt thereof, measured in in vitro settings, e.g., in 900 mL of 0.05 M pH 6.8 buffer, measured using USP Apparatus II (paddle) at 50 rpm and 37° C. In certain embodiments, compositions of the disclosure comprising cellulose acetate based polymers as water-insoluble polymers provide complete drug recovery at least about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or any intermediate periods therein, as measured in in vitro settings, e.g., in 900 mL of 0.05 M pH 6.8 buffer, measured using USP Apparatus II (paddle) at 50 rpm and 37° C.

In certain embodiments, the water-insoluble polymer is present in an amount of from about 20% to about 100%, from about 50% to about 95%, from about 60% to about 90% w/w, or any intermediate value therein, based on the total weight of the functional coat. In certain embodiments, the water-insoluble polymer is present in an amount of about 20%, about 25%, about 30%, about 35%, about 40%, about 45%, about 50%, 55%, about 60%, about 65%, about 70%, about 75%, about 80%, about 85%, about 86%, about 87%, about 88%, about 89%, about 90%, about 91%, about 92%, about 93%, about 94%, about 95%, about 100% w/w or any intermediate values therein, based on the total weight of the functional coat.

In certain embodiments, the functional coat comprises at least one anti-tacking agent. In certain embodiments, the anti-tacking agents include, but are not limited to, silicon dioxide (SYLOID® 244FP), fumed silica (CAB-O-SIL®), kaolin, talc, magnesium trisilicate, powdered starch, glyceryl monostearate and/or tribasic calcium phosphate. In certain embodiments, the anti-tacking agent can be present in an amount of from about 5% to about 30%, based on the combined weight of the water-insoluble polymer and the pore former/plasticizer.

In certain embodiments, the plasticizer acts as a pore former. In certain embodiments, the weight ratio of water-insoluble polymer and pore former/plasticizer determines the release rate of ATO or a pharmaceutically acceptable salt thereof. In certain embodiments, the range of weight ratios of water-insoluble polymer to pore former is from about 50:40 to about 99.5:0.5. In certain embodiments, the weight ratio of water-insoluble polymer to the pore former is about 50:50, about 50:40, about 55:45, about 60:40, about 65:35, about 70:30, about 75:25, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, 96:4, 97:3, 98:2, 99.5:0.5, or any intermediate ratios therein.

### 6.3. Pellets

In certain embodiments, the present disclosure provides extended release oral ATO pellet compositions that maintain solubility of the drug in different pH environments of the GI tract, and maintain a therapeutic plasma concentration of the drug for extended periods of time, without any spike or burst in release of the drug.

In certain embodiments, the pellets comprise a core comprising ATO or a pharmaceutically acceptable salt thereof and optionally, at least one organic acid. In certain embodiments, the core comprising ATO or a pharmaceutically acceptable salt thereof and, optionally, at least one organic acid is a drug layered core or a core containing the drug (e.g., drug is present in the core instead of being layered on the core). In certain embodiments, the drug layered core is a nonpareil seed/bead or a granule coated with a drug layer comprising ATO or a pharmaceutically acceptable salt thereof and, optionally, at least one organic acid. In certain embodiments the compositions of the disclosure contain a seal coat between the core and functional coat. In certain embodiments, the compositions of the disclosure contain a seal coat between the nonpareil seed/bead or a granule and the drug layer. In certain embodiments, the pellets include various components and coats in the following order: a core containing drug or coated with a drug layer; seal coat over the core and covering at least a portion of the core; and a functional coat/extended release coat over the seal coat and covering at least a portion of the seal coat.

In certain embodiments, the core is a drug layered core comprising about 49 mg of sugar sphere coated with a drug layer comprising about 100 mg of ATO HCl and about 10 mg of Hypromellose. Drug layer is coated using a solvent system comprising methanol and purified water. The drug layered core is further coated with a seal coat comprising about 1.19 mg of Hypromellose and about 3.57 mg of talc to obtain a seal coated core. The seal coated core is coated with a functional coat/ER coat comprising about 8.53 mg of cellulose acetate butyrate, about 0.84 mg of PEG 3350, and about 0.43 mg of Hypromellose. Functional coat is coated with a solvent system comprising acetone and purified water.

In certain embodiments, the core is a drug layered core comprising about 27 mg of sugar sphere coated with a drug layer comprising about 100 mg of ATO HCl, about 50 mg of fumaric acid, and about 5 mg of Hypromellose. Drug layer is coated using a solvent system comprising methanol and purified water. The drug layered core is further coated with a seal coat comprising about 1.36 mg of Hypromellose and about 4.09 mg of talc to obtain a seal coated core. The seal coated core is coated with a functional coat/ER coat comprising about 9.77 mg of cellulose acetate butyrate, about 0.97 mg of PEG 3350, and about 0.5 mg of Hypromellose. The functional coat is coated using a solvent system comprising acetone and purified water.

In certain embodiments, the core is a drug layered core comprising about 45.9 mg of sugar sphere coated with a drug layer comprising about 80 mg of ATO HCl, and about 5.5 mg of Hypromellose. Drug layer is coated using a solvent system comprising methanol and purified water. The drug layered core is further coated with a seal coat comprising about 0.99 mg of Hypromellose and about 2.96 mg of talc to obtain a seal coated core. The seal coated core is coated with a functional coat/ER coat comprising about 9.75 mg of ethyl cellulose, and about 0.98 mg of dibutyl sebacate. The functional coat is coated using a solvent system comprising isopropyl alcohol and purified water.

In certain embodiments, the core is a drug layered core comprising about 45.9 mg of sugar sphere coated with a drug layer comprising about 80 mg of ATO HCl, and about 5.53 mg of Hypromellose. Drug layer is coated using a solvent system comprising methanol and purified water. The drug layered core is further coated with a seal coat comprising about 0.99 mg of Hypromellose and about 2.96 mg of talc to obtain a seal coated core. The seal coated core is coated with a functional coat/ER coat comprising about 7.38 mg of cellulose acetate butyrate, about 1.48 mg of poly ethylene glycol (PEG 3350), and about 7.38 mg of Hypromellose phthalate (HPMC-P). The functional coat is coated using a solvent system comprising acetone and purified water.

In certain embodiments, the core is a drug layered core comprising about 48.8 mg of sugar sphere coated with a drug layer comprising about 80 mg of ATO HCl, about 8 mg of talc, and about 4 mg of Hypromellose. Drug layer is coated using a solvent system comprising methanol and purified water. The drug layered core is further coated with a seal coat comprising about 1.06 mg of Hypromellose and about 3.17 mg of talc to obtain a seal coated core. The seal coated core is coated with a functional coat/ER coat comprising about 12.08 mg of cellulose acetate butyrate, about 1.2 mg of poly ethylene glycol (PEG 3350), and about 1.2 mg of Hypromellose. The functional coat is coated using a solvent system comprising acetone and purified water.

In certain embodiments, the core is a drug layered core comprising about 48.8 mg of sugar sphere coated with a drug layer comprising about 80 mg of ATO HCl, about 8 mg of talc, and about 4 mg of Hypromellose. Drug layer is coated using a solvent system comprising methanol and purified water. The drug layered core is further coated with a seal coat comprising about 1.06 mg of Hypromellose and about 3.17 mg of talc to obtain a seal coated core. The seal coated core is coated with a functional coat/ER coat comprising about 21.77 mg of cellulose acetate butyrate, and about 4.35 mg of polyethylene glycol (PEG 3350). The functional coat is coated using a solvent system comprising acetone and purified water.

### 6.4. Methods of Making

In certain embodiments, the present disclosure provides extended release ATO compositions providing and maintaining therapeutically effective stable plasma concentrations of ATO or a pharmaceutically acceptable salt thereof, without any initial burst release/dose dumping. In certain embodiments, the compositions of the disclosure provide extended release of ATO or a pharmaceutically acceptable salt thereof for up to about 16 hours, e.g., up to about 12 hours, under physiologically relevant conditions.

The extended release ATO compositions of the disclosure include extended release pellets suitable for dosing in capsules, sachets, administration through feeding tubes, and as sprinkles on food, or liquids. In certain embodiments, the extended release pellets comprise a core that is pellet, bead/seed, or granule comprising ATO and optionally, at least one organic acid; or an inert substrate/nonpareil seed/bead or a granule coated with a coating/drug layer comprising ATO and optionally, at least one organic acid. In certain embodiments, the nonpareil beads/seeds or granules are composed of inert substrates such as starch, sugar, lactose, and/or cellulose. In certain embodiments, the nonpareil bead/seed, or a granule is a sugar sphere or microcrystalline cellulose. In certain embodiments, the core/drug layered core is coated with a seal coat covering at least a portion of the core; and a functional coat/extended release coat covering at least a portion of the seal coated core. In certain embodiments, there is no seal coat between the core and the functional coat/extended release coat.

In certain embodiments, the ATO compositions of the disclosure comprise a pellet comprising a core that is a bead/seed, or granule comprising ATO and optionally, at least one organic acid; a seal coat covering at least a portion of the core; and a functional coat/extended release coat comprising a water insoluble polymer, a pore former/plasticizer, and optionally, at least one anti-tacking agent. In certain embodiments, the presence of seal coat is optional. In certain embodiments, the core comprising ATO or a pharmaceutically acceptable salt thereof is made by wet granulation, dry granulation, hot-melt extrusion, or extrusion-spheronization. In certain embodiments, the granules are made by wet granulation process, wherein the ATO or a pharmaceutically acceptable salt thereof, and optionally, at least one organic acid are screened through mesh #40 sieve; granulated using a granulation aid to obtain granules; milled; and dried to obtain ATO granules. The ATO granules are then coated with a functional coat/extended release coat comprising a water-insoluble polymer, and at least one pore former, e.g., plasticizer or a water-soluble polymer or an enteric polymer. In certain embodiments, granules are coated with a seal coat comprising a dispersion comprising water-soluble hydrophilic polymer, a plasticizer, and talc to obtain seal coated granules. In certain embodiments, the seal coated granules are coated with a functional coat/extended release coat comprising a water-insoluble polymer, and at least one pore former.

In certain embodiments, the core is a nonpareil bead/seed, or a granule coated with a drug layer comprising atomoxetine or a pharmaceutically acceptable salt thereof and, optionally, at least one acidifier/organic acid. In certain embodiments, atomoxetine compositions are made by coating a nonpareil bead/seed, or a granule with a drug layer comprising atomoxetine or a pharmaceutically acceptable salt thereof to obtain a drug layered core; coating the drug layered core with a seal coat comprising water-soluble polymer to obtain a seal coated core; and coating the seal coated core with a functional coat/extended release coat with comprising a water-insoluble polymer, and at least one pore former, e.g., water soluble plasticizer or a water-soluble polymer or an enteric polymer.

In certain embodiments, the core is an organic acid bead/seed or granule coated with a drug layer comprising atomoxetine. In certain embodiments there is a seal coat between the organic acid granules and the drug layer. In certain embodiments, the drug layer further contains at least one organic acid. In certain embodiments, the additional organic acid in the coating can be the same as the organic acid of the organic acid granules. In certain embodiments, the additional organic acid in the coating can be different from the organic acid of the organic acid granules. In certain embodiments, atomoxetine compositions are made by coating an organic acid bead/seed or granule with a drug layer comprising atomoxetine or a pharmaceutically acceptable salt thereof to obtain a drug layered core; coating the drug layered core with a seal coat comprising water soluble polymer to obtain a seal coated core; and coating the seal coated core with a functional cot/extended release coat with comprising a water-insoluble polymer, and at least one pore former, e.g., water-soluble plasticizer or a water-soluble polymer or an enteric polymer.

In certain embodiments, there is no seal coat on the drug layered granule.

### 6.5. Methods of Treatment, Dosage, and Administration

The present disclosure provides ATO HCl extended release capsule compositions, with each capsule comprising ATO HCl equivalent to 25 mg, 40 mg, 60 mg, 80 mg, and 100 mg of ATO. In certain embodiments, the disclosure provides methods for treating symptoms of ADHD/ADD for which ATO is an appropriate treatment. In particular, the present disclosure provides methods for treating ADHD/ADD in children, adolescents age 6-18, and adults. The methods comprise once-a-day oral administration of an extended release ATO composition of the disclosure. In certain embodiments, the ATO compositions of the disclosure are administered early in the morning, with or without food, to an individual suffering from ADHD/ADD to provide calming and focusing effects throughout the day, while substantially reducing insomnia. Atomoxetine compositions of the disclosure are indicated as an integral part of a total treatment program for ADHD that may include other measures (psychological, educational, social) for patients with this syndrome. Drug treatment may not be indicated for all patients with this syndrome. Drug treatment is not intended for use in the patient who exhibits symptoms secondary to environmental factors and/or other primary psychiatric disorders, including psychosis. Appropriate educational placement is essential in children and adolescents with this diagnosis and psychosocial intervention is often helpful. When remedial measures alone are insufficient, the decision to prescribe drug treatment medication will depend upon the physician's assessment of the chronicity and severity of the patient's symptoms.

In certain treatment of ADHD/ADD with stimulants helps to reduce symptoms of these and other attention disorders and improve self-esteem, cognition, and social and family interactions of the patient. Commonly prescribed stimulants for ADHD/ADD include mixed amphetamines and methylphenidate. However stimulant medications have serious risk of drug abuse and drug dependence. ATO is a commonly used non-stimulant medication for ADHD/ADD. ATO produces calming and focusing effects on an individual suffering from ADHD/ADD, with minimal risk of drug abuse and drug dependence. Atomoxetine, a highly specific inhibitor of the parasynaptic norepinephrine transporter, is an FDA-approved treatment for ADHD in adults and children. Atomoxetine is not a psychostimulant, which sets it apart from the common medications used for ADHD, e.g., methylphenidate and mixed amphetamines. The use of pharmaceutically acceptable salts of ATO, such as ATO HCl, are also contemplated in the present disclosure.

In certain embodiments, the disclosure provides a method for improving patient compliance by administering the extended release ATO compositions of the disclosure, wherein the composition provides a reduced Cₘₐₓ, and reduced dose related peak-to-trough fluctuations, as compared to marketed IR ATO capsules.

In certain embodiments, the disclosure provides methods for reducing side effects associated with currently marketed immediate release ATO compositions. In certain embodiments, the methods comprise administering extended release ATO compositions of the disclosure that (1) reduce initial burst release/dose dumping and (2) maintain therapeutic plasma concentrations of the drug for extended periods of time, e.g., up to about 16 hours. In certain embodiments, the therapeutic plasma concentrations depend on the severity of the patient's condition, and on the strength of the ATO composition administered to the patient. In certain embodiments, the compositions of the disclosure contain from about 40% w/w to about 80% w/w, based on the total weight of the composition, of ATO HCl.

In certain embodiments, the disclosure provides methods for improving patient compliance by administering extended release ATO compositions of the disclosure, wherein the extended release compositions will allow for reduced frequency of administration of the composition, administration as sprinkle on solid food or liquids, administration via feeding tubes, and reduce side effects associated with high Cₘₐₓ levels. In certain embodiments, the compositions of the disclosure reduce or avoid initial burst release (Cₘₐₓ above the therapeutic range) while providing therapeutically effective amounts of ATO HCl for periods of up to about 16 hours.

In certain embodiments, the disclosure provides methods for improving patient compliance by administering extended release ATO compositions that reduce or avoid an initial burst release of atomoxetine hydrochloride, suitable for once-daily administration, and provide desired therapeutic effects with minimal side effects. In certain embodiments, the ATO compositions of the disclosure reduce side effects in children, such as nausea, vomiting, fatigue, and abdominal pain, which side effects are associated with once-daily administration of marketed immediate release atomoxetine compositions that provide high peak serum concentrations (Cₘₐₓ) and low trough-to-peak concentration ratios. In certain embodiments, the ATO compositions of the disclosure reduce side effects in adults, such as constipation, dry mouth nausea, palpitations, decreased appetite, insomnia, libido disease, sleep disorder, somnolence, decreased appetite, dizziness, erectile dysfunction, and urinary hesitation (renal and urinary disorders), vascular disorders (e.g., hot flush), and skin and subcutaneous tissue disorders (e.g., hyperhidrosis), which side effects are associated with once-daily administration of marketed immediate release atomoxetine compositions that exhibit high peak serum concentrations (Cₘₐₓ) and high peak-to-trough concentration ratios. The methods comprise administering to the patient extended release ATO compositions of the disclosure.

In certain embodiments, extended release atomoxetine compositions of the disclosure, suitable for once-daily administration, improve patient adherence, reduce stigma (because child or young person does not need to take medication at school), reduce problems associated with storing and administering controlled drugs, and improve PK profiles by reducing peak plasma concentrations (Cₘₐₓ), and peak-to-trough fluctuations.

In certain embodiments, the disclosure provides extended release ATO compositions, wherein the compositions minimize alcohol induced dose dumping. Alcohol induced dose dumping (AIDD) can be defined as the rapid unintended release of a large amount of drug from an extended release dosage form resulting from an accidental misuse or from intentional abuse of alcohol with the drug. Such rapid drug release from an extended release dosage form, i.e., dose dumping, results in the administration of a single bolus dose leading to increased exposure levels, possible safety issues and adverse events. This situation is of most concern for centrally acting drugs with high bioavailability. It has been found that presence of water-insoluble cellulose acetate based polymers in the extended release coatings of the compositions of the disclosure substantially reduce alcohol induced dose dumping of atomoxetine or a pharmaceutically acceptable salt thereof. In certain embodiments, presence of cellulose acetate butyrate in the functional coat substantially reduces alcohol induced dose dumping of atomoxetine or a pharmaceutically acceptable salt thereof.

The following Examples illustrate the disclosure in a nonlimiting manner. Unless indicated to the contrary, the numerical parameters set forth herein can vary depending upon the desired properties sought to be obtained by the present disclosure.

### 7. EXAMPLES

### Example 1: Preparation of Extended Release ATO HCl Pellets

The present Example provides a summary of the preparation of two different extended release atomoxetine compositions comprising Pellets Type 1 (Pellet 1) and Pellets Type 2 (Pellet 2) as shown in Table 1.

TABLE 1

| Composition | Pellet 1 (mg/dose) | Pellet 2 (mg/dose) |
|---|---|---|
| Core | | |
| Sugar sphere | 48.70 | 26.70 |
| Drug layer | | |
| Atomoxetine HCl | 100.0 | 100.0 |
| Hypromellose 5 mpas | 10.0 | 5.0 |
| Fumaric acid | NA | 50.0 |
| Solvent System* | Methanol:Purified Water (90:10) | |
| Total Drug Layer Weight | 110.0 | 155.0 |
| Total weight of drug | 158.70 | 181.70 |
| Layered core | | |
| Seal Coat | | |
| Hypromellose | 1.19 | 1.36 |
| Talc | 3.57 | 4.09 |
| Solvent System* | Ethanol:Purified Water (75:25) | |
| Total Seal Coat Weight | 4.76 | 5.45 |
| Functional Coat/ER Coat | | |
| Cellulose acetate butyrate | 8.53 | 9.77 |
| (CAB) | | |
| Polyethylene glycol (PEG | 0.84 | 0.97 |
| 3350) | | |
| Hypromellose | 0.43 | 0.49 |
| Solvent System* | Acetone:Purified Water (90:10) | |
| Total Functional Coat | 9.81 | 11.23 |
| Weight | | |
| Total Weight | 173.27 | 198.38 |

*Removed during process

The pellets were made according to the following manufacturing procedure.

### Manufacturing Procedure:

### A. Drug Layering:

A-1: Hypromellose was added to a mixture of methanol and water in a stainless-steel container and mixed until a clear solution was obtained.

A-2: To the clear solution from step #A-1, ATO HCl was added and mixed until completely dissolved to obtain drug layering solution.

A-3 Fumaric acid was added to the drug layering solution from step #A-2 and mixed for not less than 15 minutes.

A-4: Sugar spheres were coated with drug layering solution form step #A-2 with a coating weight gain of approximately 226% w/w for Pellets Type 1 (Pellet 1); and with drug layering dispersion from step #A-3 with a coating weight gain of approximately 580% w/w for Pellets Type 2 (Pellet 2).

### B. Seal Coating:

B-1: Hypromellose was dissolved in ethanol-water solvent system to obtain a clear solution.

B-2: To the clear solution from step #B-1, talc was added and the resulting mixture was mixed for not less than 15 minutes.

B-3: Drug layered Pellets Type 1 (drug layered Pellet 1) from step #A were coated with the seal coating dispersion from step #B-2 to obtain seal coated Pellets Type 1 (seal coated Pellet 2); and drug layered Pellets Type 2 (drug layered Pellet 2) from step #A were coated with the seal coating dispersion from step #B-2 to obtain seal coated Pellets Type 2 (seal coated Pellet 2).

B-4: Seal coated pellets Type 1 and Type 2 from step #B-3 were dried.

### C. Functional Coat/ER Coat:

C-1: Cellulose acetate butyrate was dissolved in acetone-water solvent system.

C-2: To the solution from step #C-1, PEG was added and mixed to obtain a clear solution.

C-3: Hypromellose solution in water was prepared separately and added to the clear solution from step #C-2.

C-4: Seal coated pellets Type 1 (seal coated Pellet 1) 1 from step B were coated with the solution from step #C-3 to obtain functional coated Pellets Type 1 (Pellet 1); and seal coated pellets Type 2 (seal coated Pellet 2) from step B were coated with the solution from step #C-3 to obtain functional coated pellets Type 2 (Pellet 2).

### D. ATO HCl Capsules:

D-1: A final blend of functional coated pellets from step C along with talc was prepared using a V-Blender and then filled into hard gelatin capsules based on the required fill weight.

### Example 2: Preparation of Extended Release ATO HCl Pellets

The present Example provides a summary of the preparation of 8 different extended release atomoxetine compositions comprising pellets Type 3-10 (Pellets 3-10) shown in Table 2 and Table 3.

TABLE 2

| Composition | Pellet 3 | Pellet 4 | Pellet 5 | Pellet 6 |
|---|---|---|---|---|
| | Granulated | Granulated | Granulated | Core |
| | Core (wt. %) | Core (wt. %) | Core (wt. %) | (wt. %) |
| Sugar Sphere | — | — | — | 26 |
| | — | — | — | Drug Layer |
| | | | | (wt. %) |
| Atomoxetine | 75 | 55 | 52 | 51 |
| HCl | | | | |
| Hypromellose | 5 | 5 | — | — |
| 5 mpas | | | | |
| Fumaric acid | 20 | 20 | | 21 |
| Tartaric acid | — | — | 20 | — |
| Microcrystalline | — | 20 | 15 | — |
| cellulose | | | | |
| Kollidon | — | — | 3 | 2 |
| Talc | — | — | 10 | — |
| Total Core/Drug | 100 | 100 | 100 | 100 |
| Layered Core | | | | |
| (wt. %) | | | | |
| Functional | | | | |
| Coat/ER Coat | | | | |
| Cellulose acetate | — | 37.25 | 90 | — |
| butyrate | | | | |
| Cellulose acetate | — | — | — | — |
| Ethyl Cellulose | 84 | — | — | — |
| EUDRAGIT ® | — | 37.25 | — | 31.31 |
| RS | | | | |
| EUDRAGIT ® | — | — | — | 31.31 |
| RL | | | | |
| PEG 400 | — | 11.76 | 10 | — |
| Dibutyl sebacate | 8 | — | — | — |
| Triethyl citrate | — | — | — | 6 |
| Talc | 8 | 13.72 | — | 31.31 |
| Total Functional | 100 | 100 | 100 | 100 |
| Coat (wt. %) | | | | |

TABLE 3

| Composition | Pellet 7 | Pellet 8 | Pellet 9 | Pellet 10 |
|---|---|---|---|---|
| | Core | Granulated | Core | Core |
| | (wt. %) | Core (wt. %) | (wt. %) | (wt. %) |
| Tartaric Acid | 36 | — | — | — |
| Microcrystalline | — | — | 18.5 | |
| cellulose | | | | |
| | Drug layer | | Drug Layer | Drug layer |
| | (wt %) | | (wt. %) | (wt. %) |
| Atomoxetine | 62.5 | 52 | 52 | 52 |
| HCl | | | | |
| Hypromellose | — | — | — | — |
| 5 mpas | | | | |
| Fumaric acid | — | — | — | — |
| Tartaric acid | | 20 | 19 | 19 |
| Microcrystalline | — | 15 | — | 18.5 |
| cellulose | | | | |
| Kollidon | 1.5 | 3 | 3.5 | 3.5 |
| Talc | | 10 | 7 | 7 |
| Total Core/Drug | 100 | 100 | 100 | 100 |
| layered Core | | | | |
| (wt. %) | | | | |
| Functional | | | | |
| Coat/ER Coat | | | | |
| Cellulose acetate | — | — | — | — |
| butyrate | | | | |
| Cellulose acetate | — | 90 | — | — |
| Ethyl Cellulose | — | — | — | 80 |
| EUDRAGIT ® | — | — | — | — |
| RS | | | | |
| EUDRAGIT ® | — | — | — | — |
| RL | | | | |
| EUDRAGIT ® | — | — | 56.6 | — |
| NE | | | | |
| Shellac | 83.33 | — | | — |
| Hypromellose | — | — | 18.8 | — |
| 5 mpas | | | | |
| PEG 400 | — | 10 | — | — |
| Dibutyl sebacate | — | — | — | 8 |
| Triethyl citrate | 4.165 | — | — | — |
| Polysorbate 80 | — | — | 5.6 | — |
| Talc | 4.165 | — | 18.8 | 12 |
| Kollidon | 8.33 | — | — | — |
| Total Functional | 100 | 100 | 100 | 100 |
| Coat (wt. %) | | | | |

### Pellet 3:

### Manufacturing Procedure:

### A. Pellet Core:

A-1: ATO HCl and fumaric acid are screened through mesh #40 sieve.

A-2: Hypromellose is dissolved in purified water to provide granulation aid.

A-3: The ATO HCl and fumaric acid mixture from step #A-1 is granulated using granulation aid from step #A-2.

A-4: Granules from step #A-3 are milled and dried to obtain pellet cores.

### B. Functional Coat/ER Coat:

Pellet cores from step A are coated with functional coat comprising ethyl cellulose, dibutyl sebacate and talc, using the functional coating process described in Example, 1 to obtain functional coated Pellets Type 3 (Pellet 3).

### Pellet 4:

### A. Pellet Core:

A-1: ATO HCl, fumaric acid, and microcrystalline cellulose are screened through mesh #40 sieve.

A-2: Hypromellose is dissolved in purified water to provide granulation aid.

A-3: The ATO HCl, fumaric acid, and microcrystalline cellulose mixture from step #A-1 is granulated using granulation aid from step #A-2.

A-4: The wet-granulated mass from step #A-3 is passed through an extruder; the obtained extrudate is then processed using a spheronizer to form pellet cores.

A-5: Pellet cores from step #A-4 are dried.

### B. Functional Coat/ER Coat:

Pellet cores from step A are coated with functional coat comprising cellulose acetate butyrate, EUDRAGIT© RS, PEG 400, and talc, using the functional coating process described in Example 1, to obtain functional coated Pellets Type 4 (Pellet 4).

### Pellet 5:

### A. Pellet Core:

A-1: ATO HCl, tartaric acid, microcrystalline cellulose, and talc are screened through mesh #40 sieve.

A-2: Kollidon is dissolved in ethanol-water solvent system to provide granulation aid.

A-3: The ATO HCl, tartaric acid, microcrystalline cellulose, and talc mixture from step #A-1 is granulated using granulation aid from step #A-2.

A-4: Granules from step #A-3 are milled and dried to obtain pellet cores.

### B. Functional Coat/ER Coat:

Pellet cores from step A are coated with functional coat comprising cellulose acetate butyrate, and PEG 400, using the functional coating process described in Example 1, to obtain functional coated Pellets Type 5 (Pellet 5).

### Pellet 6:

### A. Drug Layering:

A-1: ATO HCl, fumaric acid, and kollidon are dispersed in ethanol-Isopropyl alcohol solvent system.

A-2: Sugar spheres are coated with drug layering dispersion form step #A-1 using a rotor granulator.

A-3: Drug layered pellets are dried until the solvent is removed.

### B. Functional Coat/ER Coat:

C-1: Drug layered pellets from step A are coated with functional coat comprising EUDRAGIT® RS, EUDRAGIT® RL, triethyl citrate and talc, using the functional coating process described in Example 1, to obtain functional coated Pellets Type 6 (Pellet 6).

### Pellet 7:

### A. Drug Layering:

A-1: ATO HCl and Kollidon are dispersed in ethanol-Isopropyl alcohol solvent system.

A-2: Tartaric acid pellets are coated with drug layering solution form step #A-1 using a rotor granulator to obtain drug layered pellets.

A-3: Drug layered pellets from step #A-2 are dried until the solvent is removed.

### B. Functional Coat/ER Coat:

C-1: Drug layered pellets from step #A-3 are coated with functional coat comprising shellac, Kollidon, triethyl citrate and talc, using the functional coating process described in Example 1, to obtain functional coated Pellets Type 7 (functional coated Pellet 7).

### Pellet 8:

### A. Pellet Core:

A-1: ATO HCl, microcrystalline cellulose, tartaric acid and talc are screened through mesh #40 sieve.

A-2: Kollidon is dissolved in ethanol-purified water solvent system to provide granulation aid.

A-3: The ATO HCl microcrystalline cellulose, tartaric acid and talc mixture from step #A-1 is granulated using granulation aid from step #A-2 using a rotor granulator to obtain pellet cores.

A-4: Pellet cores obtained from step #A-3 are dried until the solvent is removed.

### B. Functional Coat/ER Coat:

Pellet cores from step A are coated with functional coat comprising cellulose acetate and polyethylene glycol, using the functional coating process described in Example 1, to obtain functional coated Pellets Type 8 (Pellet 8).

### Pellet 9:

### A. Drug Layering:

A-1: ATO HCl, tartaric acid, talc, and kollidon are dispersed in ethanol-Isopropyl alcohol solvent system.

A-2: Microcrystalline cellulose spheres are coated with drug layering solution form step #A-1 using a rotor granulator to obtain drug layered pellets.

A-3: Drug layered pellets are dried until the solvent is removed.

### B. Functional Coat/ER Coat:

B-1: Pellet cores from step A are coated with functional coat comprising EUDRAGIT© NE 30D, Hypromellose, Polysorbate 80, and talc, using the functional coating process described in Example 1, to obtain functional coated Pellets Type 9 (Pellet 9).

### Pellet 10:

### A. Drug Layering:

A-1: ATO HCl, tartaric acid, talc, and Kollidon are dispersed in ethanol-Isopropyl alcohol solvent system.

A-2: Microcrystalline cellulose spheres are coated with drug layering solution form step #A-1 using a rotor granulator to obtain drug layered pellets.

A-3: Drug layered pellets are dried until the solvent is removed.

### B. Functional Coat/ER Coat:

B-1: Drug layered pellets from step A are coated with functional coat comprising ethyl cellulose, dibutyl sebacate, and talc, using the functional coating process described in Example 1, to obtain functional coated Pellets Type 10 (Pellet 10).

### Example 3: Comparison of Atomoxetine Hydrochloride Release Profile from Strattera (60 mg), Compositions Comprising Pellet 1, and Compositions Comprising Pellet 2, and Under Different pH Conditions

Dissolution tests were performed for Strattera (60 mg), composition comprising Pellet 1, and composition comprising Pellet 2. Pellet 1 is drug layered Pellet without any acid in the pellet core. Pellet 2 is a drug layered pellet containing fumaric acid. The dissolutions were performed using USP Apparatus 2 (Paddle), with sinkers, in 900 mL of a dissolution medium comprising 0.1 N HCl, pH 4.5 acetate buffer, pH 6.8 phosphate buffer, and pH 7.5 phosphate buffer, at 37° C. and 50 rpm.

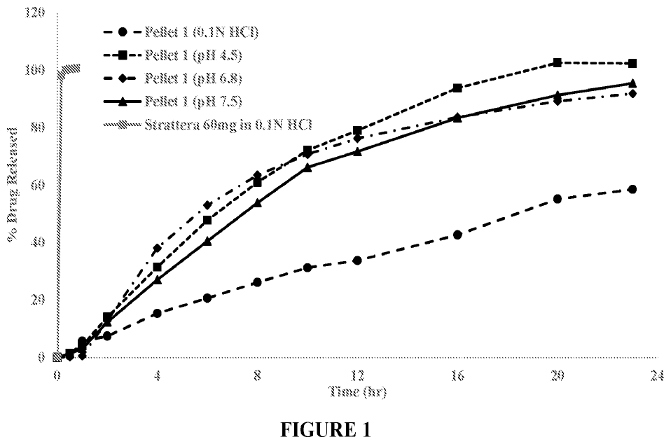

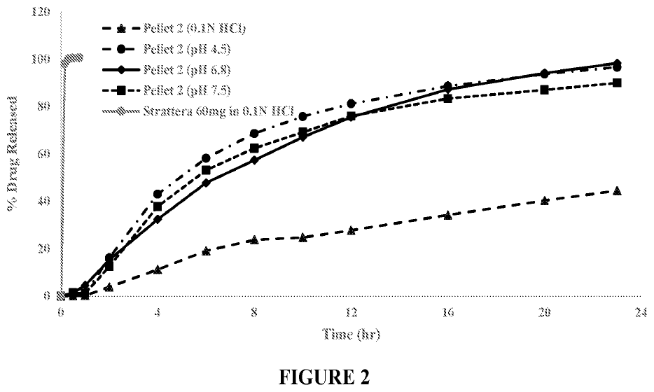

FIGS. 1 and 2 show that composition comprising Pellet 1 and composition comprising Pellet 2, each provides an extended release profile with substantially reduced initial plasma concentrations of ATO HCl compared to Strattera. Example 4: Additional compositions Comprising ATO HCl pellets

The present Example provides a summary of the preparation of 4 different extended release atomoxetine compositions comprising Pellets Type 11-14 (Pellets 11-14), as shown in Table 4.

TABLE 4

| | Pellet 11 | Pellet 12 | Pellet 13 | Pellet 14 |
|---|---|---|---|---|
| Composition | (mg/dose) | (mg/dose) | (mg/dose) | (mg/dose) |
| Core | | | | |
| Sugar Sphere | 45.92 | 45.92 | 48.88 | 48.88 |
| Drug layer | 186% Coating | 186% Coating | 188% Coating | 188% Coating |
| | wt. gain | wt. gain | wt. gain | wt. gain |
| Atomoxetine | 80 | 80 | 80 | 80 |
| HCl | | | | |
| Hypromellose | 5.53 | 5.53 | 4 | 4 |
| 5 mpas | | | | |
| Talc | — | — | 8 | 8 |
| Solvent | Methanol:Purified | Methanol:Purified | Methanol:Purified | Methanol:Purified |
| System* | Water (90:10) | Water (90:10) | Water (90:10) | Water (90:10) |
| Total Drug | 85.53 | 85.53 | 92 | 92 |
| Layer wt. | | | | |
| Total Weight | 131.45 | 131.45 | 140.88 | 140.88 |
| of Drug | | | | |
| Layered Core | | | | |
| Seal Coat | 3% Coating | 3% Coating | 3% Coating | 3% Coating |
| | wt. gain | wt. gain | wt. gain | wt. gain |
| Hypromellose | 0.99 | 0.99 | 1.06 | 1.07 |
| 5 mpas | | | | |
| Micro Talc | 2.96 | 2.96 | 3.17 | 3.18 |
| Total Seal | 3.94 | 3.94 | 4.23 | 4.25 |
| Coat Weight | | | | |
| Solvent | Ethanol:Purified | Ethanol:Purified | Ethanol:Purified | Ethanol:Purified |
| System* | Water (75:25) | Water (75:25) | Water (95:5) | Water (95:5) |
| Functional | | | | |
| Coat/ER Coat | | | | |
| Ethyl Cellulose | 9.75 | — | — | — |
| Dibutyl | 0.98 | — | — | — |
| sebacate | | | | |
| CAB | — | 7.38 | 12.08 | 21.77 |
| (Cellulose | | | | |
| acetate | | | | |
| butyrate) | | | | |
| PEG 3350 | — | 1.48 | 1.20 | 4.35 |
| (Polyethylene | | | | |
| Glycol) | | | | |
| HPMC—P | — | 7.38 | | — |
| (Hypromellose | | | | |
| Phthalate) | | | | |
| Hypromellose | | | 1.20 | |
| Total | 10.73 | 16.24 | 14.48 | 26.12 |
| Functional | | | | |
| Coat Weight | | | | |
| Solvent | Isopropyl | Acetone:Purified | Acetone:Purified | Acetone:Purified |
| System* | alcohol:Purified | Water (95:5) | Water (90:10) | Water (90:10) |
| | Water (95:5) | | | |
| Total Weight | 146 | 152 | 160 | 171 |

*Removed during process

The pellets were made according to the following manufacturing procedure.

### Manufacturing Procedure:

### A. Drug Layering:

A-1: Hypromellose was added to a mixture of methanol and water in a stainless-steel container and mixed until a clear solution was obtained.

A-2: To the clear solution from step #A-1, ATO HCl was added and mixed until completely dissolved to obtain drug layering solution.

A-3: For Pellets Type 13 and Pellets Type 14, talc was added to the drug layering solution from Step #A-2.

A-4: Sugar spheres were coated with drug layering solution form step #A-2 with a coating weight gain of approximately 186% w/w to obtain drug layered Pellets Type 11 and Pellets Type 12 (drug layered Pellet 11, 12); sugar spheres were coated with drug layering solution from step #A-3 with a coating weight gain of approximately 188% w/w to obtain drug layered Pellets Type 13 and 14 (drug layered Pellet 13 and Pellet 14).

### B. Seal Coating:

B-1: Hypromellose was dissolved in ethanol-water solvent system to obtain a clear solution.

B-2: To the clear solution from step #B-1, talc was added and the resulting mixture was mixed for not less than 15 minutes.

B-3: Drug layered Pellets Type 11, 12, 13, and 14 (drug layered Pellet 11-14) from step #A were coated with the seal coating dispersion from step #B-2 to obtain seal coated Pellets Type 11-14 (seal coated Pellets 11-14).

B-4: Seal coated pellets 11-14 from step #B-3 were dried.

### C. Functional Coat/ER Coat:

### Pellet 11:

C-1: Ethyl cellulose was dissolved in isopropyl alcohol-water solvent system.

C-2: To the solution from step #C-1, dibutyl sebacate was added and mixed to obtain a clear solution.

C-3: Seal coated pellets Type 11 (seal coated Pellet 11) 1 from step B were coated with the solution from step #C-2 to obtain functional coated Pellets Type 11 (Pellet 11).

### Pellet 12:

C-1: Cellulose acetate butyrate (CAB 171-NF) and Hypromellose phthalate (HPMC-P) were added to acetone-water solvent system and mixed until completely dissolved.

C-2: To the solution from step #C-1, polyethylene glycol (PEG 3350) was added and mixed to obtain a clear solution.

C-3: Seal coated pellets Type 12 (seal coated Pellet 12) 1 from step B were coated with the solution from step #C-2 to obtain functional coated Pellets Type 12 (Pellet 12).

### Pellet 13:

C-1: Hypromellose (Hypromellose 5 mpas) was added to water and mixed until completely dissolved to obtain Hypromellose solution.

C-2: Hypromellose solution from step #C-1 was added to acetone and mixed until completely miscible.

C-3: Polyethylene glycol (PEG 3350) was added to the solution from step #C-2 and mixed until a clear solution was obtained.

C-4: Seal coated pellets Type 13 (seal coated Pellet 13) 1 from step B were coated with the solution from step #C-3 to obtain functional coated Pellets Type 13 (Pellet 13).

### Pellet 14:

C-1: Acetone and purified water were mixed to obtain a solvent mixture.

C-2: Cellulose acetate butyrate (CAB 171-NF) was added to the solvent mixture from step #C-1 and mixed for not less than 15 minutes or until completely soluble.

C-3: To the solution from step #C-2, polyethylene glycol (PEG 3350) was added and mixed to obtain a clear solution.

C-4: Seal coated pellets Type 14 (seal coated Pellet 14) 1 from step B were coated with the solution from step #C-3 to obtain functional coated Pellets Type 14 (Pellet 14).

### D. ATO HCl Capsules:

D-1: Extended release pellets equivalent to 80 mg of Atomoxetine HCl (ATO HCl) from step C (Pellets 11-14) were filled into hard gelatin capsules.

### Example 5: Comparison of Atomoxetine Hydrochloride Release Profile from Compositions Comprising Pellet 11 at Different Coating Weight Gains

Dissolution tests were performed for composition comprising Pellet 11a with 6% functional coating weight gain, and composition comprising Pellet 11 b with 8% functional coating weight gain. Pellets 11.a and 11. b contained functional coat comprising ethyl cellulose and dibutyl sebacate in a wt. ratio of about 91:9. The dissolutions were performed using USP Apparatus 2 (Paddle), with sinkers, in 900 mL of a dissolution medium comprising 0.05 M of pH 6.8 buffer, at 37° C. and 50 rpm.

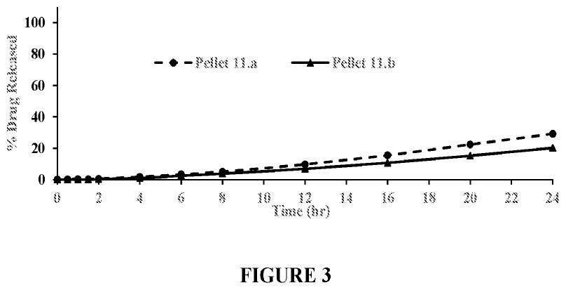

FIG. 3 shows an initial delay in release of Atomoxetine HCl for up to 4 hours and drug recovery of about 29% at 24 hours for Pellet 11.a with 6% w/w coating weight gain, and 20% drug release at 24 hours for Pellet 11.b with 8% w/w coating weight gain. Extended release coating comprising ethyl cellulose and dibutyl sebacate did not provide drug recovery of more than 29% at 24 hours.

### Example 6: Comparison of Atomoxetine Hydrochloride Release Profile, from Compositions Comprising Pellet 12 at Different Coating Weight Gains, in Three Stage Dissolution Media

Dissolution tests were performed for composition comprising Pellet 12.a with 6% functional coating weight gain, and composition comprising Pellet 12.b with 8% functional coating weight gain. Pellets 12.a and 12.b contained functional coat comprising cellulose aetate butyrate (CAB 171-15 NF), polyethylene glycol (PEG-3350) and hydroxypropyl methylcellulose phthalate (HPMC-P) in a wt. ratio of about 5:1:5. The dissolutions were performed using USP Apparatus 2 (Paddle), with sinkers, in three stage dissolution media comprising 500 mL of 0.01N HCl (0-1 hr); 750 mL of pH 4.5 acetate buffer (1-2 hrs); and then in 1000 mL of pH 6.8 buffer (2-24 hrs), at 37° C. and 50 rpm. Three stage dissolutions were performed as follows:

Stage 1: Pellets were added to 500 mL of 0.01N HCl (0-1 hrs).

Stage 2: At 1 hr, to above vessels, added 250 mL of pH 4.98, 0.1M acetate buffer and then adjusted with NaOH or HCl until desired pH is reached (1-2 hrs).

Stage 3: At 2 hrs, to above vessels, added 250 mL of pH 10.76, 0.1M phosphate buffer and then adjusted with NaOH or HCl until desired pH is reached (2-24 hrs).

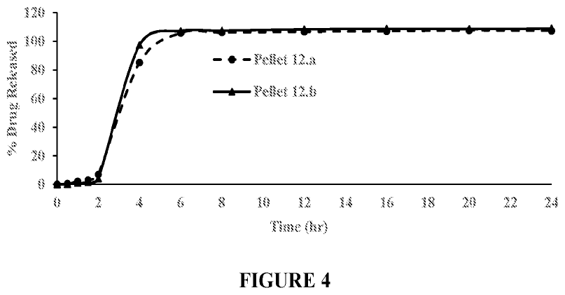

FIG. 4 shows a delay in release of Atomoxetine HCl from the pellets for first 90 minutes followed by complete release by 4 hours. This could be because of the HPMC-P in the ER coating layer which has pH dependent solubility and completely dissolves above pH 5-5.5.

### Example 7: Comparison of Atomoxetine Hydrochloride Release Profile from Compositions Comprising Pellet 13 at Different Coating Weight Gains

Dissolution tests were performed for composition comprising Pellet 13.a with 6% functional coating weight gain, and composition comprising Pellet 13.b with 8% functional coating weight gain. Pellets 13.a and 13.b contained functional coat comprising cellulose aetate butyrate (CAB 171-15 NF), polyethylene glycol (PEG-3350) and hydroxypropyl methylcellulose (HPMC) in a wt. ratio of about 10:1:1. The dissolutions were performed using USP Apparatus 2 (Paddle), with sinkers, in 900 mL of a dissolution medium comprising 0.05 M of pH 6.8 buffer, at 37° C. and 50 rpm.

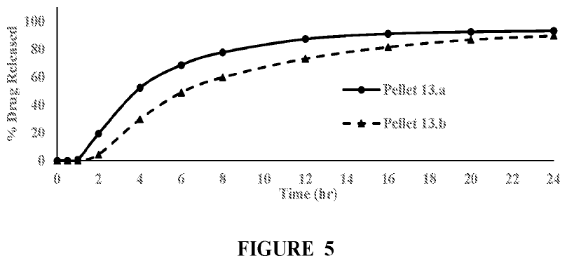

FIG. 5 shows an initial delay in release of Atomoxetine HCl followed by gradual release extending up to 12 hours for Pellet 13.a with 6% w/w coating weight gain and up to 20 hours for Pellet 13.b with 10% w/w coating weight gain.

### Example 8: Comparison of Atomoxetine Hydrochloride Release Profile from Compositions Comprising Pellet 14 at Different Coating Weight Gains

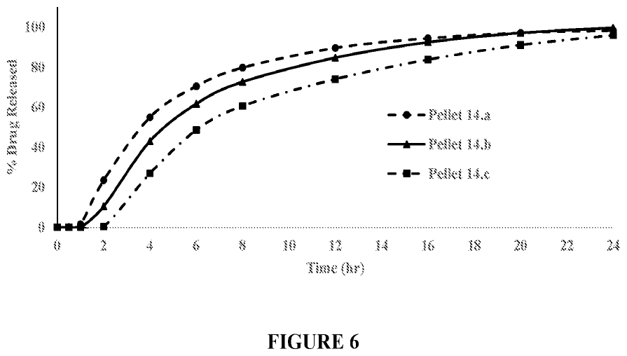

Dissolution tests were performed for composition comprising Pellet 14.a with 6% functional coating weight gain, composition comprising Pellet 14.b with 8% functional coating weight gain, and composition comprising Pellet 14.c with 18% functional coating weight gain. Pellets 14.a, 14.b, and 14.c contained functional coat comprising cellulose aetate butyrate (CAB 171-15 NF) and polyethylene glycol (PEG-3350) in a wt. ratio of about 5:1. The dissolutions were performed using USP Apparatus 2 (Paddle), with sinkers, in 900 mL of a dissolution medium comprising 0.05 M of pH 6.8 buffer, at 37° C. and 50 rpm. FIG. 6 shows an initial delay in release of Atomoxetine HCl in first 1-2 hours, followed by extended release for about 12 hours for pellets with 6% coating weight gain, about 16 hours for pellets with about 8% coating weight gain, and for about 20 hours for pellets with 18% coating weight gain.

The present disclosure is well adapted to attain the ends and advantages mentioned, as well as those that are inherent therein. The particular embodiments disclosed above are illustrative only, as the present disclosure can be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above can be altered or modified, and all such variations, including but not limited to substitution of different atomoxetine salts, are considered within the scope and spirit of the present disclosure. Various publications, patents, and patent application are cited herein, the contents of which are hereby incorporated-by-reference herein in their entireties.

1. A pharmaceutical pellet composition comprising:
a) a core comprising atomoxetine or a pharmaceutically acceptable salt thereof; and
b) a functional coat covering at least a portion of the core, wherein the functional coat comprises a cellulose acetate-based polymer selected from the group consisting of cellulose acetate, cellulose acetate butyrate, cellulose diacetate, cellulose triacetate, cellulose acetate phthalate or mixtures thereof, and optionally a pore former and/or a plasticizer,
wherein the composition releases less than or equal to 40% of the atomoxetine or the pharmaceutically acceptable salt thereof, based on the total weight of atomoxetine or the pharmaceutically acceptable salt thereof present in the composition, within 2 hours of coming in contact with 900 mL of a dissolution medium comprising 0.05 M pH 6.8 buffer, measured using USP Apparatus II (paddle) at 50 rpm and 37° C.

2. The pharmaceutical pellet composition of claim 1,
wherein the composition provides a lag time of between about 30 minutes and about 2 hours, during which the composition releases less than or equal to 10% of atomoxetine or a pharmaceutically acceptable salt thereof, measured using USP Apparatus 2 (Paddle), with sinkers, in 900 mL of a dissolution medium comprising 0.05 M of pH 6.8 buffer, at 37° C. and 50 rpm.

3.-4. (canceled)

5. The composition of claim 1, wherein the atomoxetine or the pharmaceutically acceptable salt thereof is present in an amount of from about 20% w/w to about 80% w/w, based on the total weight of the composition.

6. The composition of claim 1, wherein the water-insoluble polymer and the pore former are present in a weight ratio of from about 50:50 to about 99:1.

7. The composition of claim 1, wherein the water-insoluble polymer and the plasticizer are present in a weight ratio of from about 70:30 to about 99:1.

8. The composition of claim 1, wherein the core further comprises an organic acid selected from the group comprising tartaric acid, citric acid, fumaric acid, succinic acid, malic acid, maleic acid, itaconic acid, glycolic acid, and combinations thereof.

9. (canceled)

10. The composition of claim 1, wherein the plasticizer is water-soluble selected from the group comprising glycerin, polyethylene glycol monomethyl ether, triethyl citrate, triacetin, polyethylene glycol, propylene glycol, sorbitol sorbitan solution, or mixtures thereof.

11. (canceled)

12. The composition of claim 1, wherein the composition provides extended release for at least about 8 hours.

13. (canceled)

14. The composition of claim 1, wherein the core is a pellet, bead/seed, or a granule coated with a drug layer comprising atomoxetine or a pharmaceutically acceptable salt thereof.

15. The composition of claim 1, wherein the core is a pellet, bead/seed or a granule containing atomoxetine or a pharmaceutically acceptable salt thereof.

16. The composition of claim 1, wherein the pellet, bead/seed, or a granule is a nonpareil bead/seed, or a granule.

17. A method for making a pharmaceutical pellet composition comprising:
a) coating a nonpareil seed/bead, or a granule, with a drug layer comprising atomoxetine or a pharmaceutically acceptable salt thereof to obtain a drug layered core;
b) optionally, coating the drug layered core with a seal coat comprising a water soluble polymer to obtain a seal coated core; and
c) coating the core from step a) or step b) with a functional coat/extended release coat comprising a cellulose acetate-based polymer selected from the group consisting of cellulose acetate, cellulose acetate butyrate, cellulose diacetate, cellulose triacetate, cellulose acetate phthalate or mixtures thereof, and optionally at least one pore former and/or plasticizer,
wherein the composition releases less than or equal to 40% of the atomoxetine or the pharmaceutically acceptable salt thereof, based on the total weight of atomoxetine or the pharmaceutically acceptable salt thereof present in the composition, within 2 hours of coming in contact with 900 mL of a dissolution medium comprising 0.05 M pH 6.8 buffer, measured using USP Apparatus II (paddle) at 50 rpm and 37° C.

18. A method for making a pharmaceutical pellet composition comprising:
a) making a core containing atomoxetine or a pharmaceutically acceptable salt thereof using extrusion-spheronization process to obtain a drug containing core;
b) optionally, coating the drug containing core with a seal coat comprising water-soluble polymer to obtain a seal coated core; and
c) coating the core from step a) or step b) with a functional coat/extended release coat comprising a cellulose acetate-based polymer selected from the group consisting of cellulose acetate, cellulose acetate butyrate, cellulose diacetate, cellulose triacetate, cellulose acetate phthalate or mixtures thereof, and optionally at least one pore former and/or a plasticizer,
wherein the composition releases less than or equal to 40% of the atomoxetine or the pharmaceutically acceptable salt thereof, based on the total weight of atomoxetine or the pharmaceutically acceptable salt thereof present in the composition, within 2 hours of coming in contact with 900 mL of a dissolution medium comprising 0.05 M pH 6.8 buffer, measured using USP Apparatus II (paddle) at 50 rpm and 37° C.

19.-24. (canceled)

25. The pharmaceutical pellet composition of claim 1, wherein the functional coat consists of the cellulose acetate-based polymer, the pore former and the plasticizer.

26. The pharmaceutical pellet composition of claim 1, wherein the functional coat consists of the cellulose acetate-based polymer and optionally the pore former and plasticizer.

27. The pharmaceutical pellet composition of claim 1, wherein the functional coat consists of the cellulose acetate-based polymer and the plasticizer.

28. The pharmaceutical pellet composition of claim 1, wherein the functional coat consists of the cellulose acetate-based polymer and the pore former.

29. The composition of claim 1, wherein the pore former is selected from the group consisting of hydroxypropyl cellulose, hydroxypropyl methyl cellulose and polyvinylpyrrolidone, or mixtures thereof.

30. A capsule comprising the pellet of claim 1.

31. A method of treating attention deficit hyperactivity disorder in a subject, the method comprising administering the capsule of claim 30.
